(19) United States
(12) Patent Application Publication
(10) Pub. No.: US 2026/0183273 A1
(43) Pub. Date: Jul. 2, 2026

(54) N,N-dialkyl-4-(2-ethylindan-2-yl)-1H-imidazole-1-carboxamides and related compounds for treatment of neurodegenerative diseases

(71) Applicant: Univerza v Ljubljani, 1000 Ljubljana (SI)

(72) Inventors: Anze Meden, Ljubljana (SI); Damijan Knez, Ljubljana (SI); Stanislav Gobec, Ljubljana (SI)

(21) Appl. No.: 19/132,010

(22) PCT Filed: Jan. 31, 2024
(86) PCT No.: PCT/EP2024/052396

§ 371 Date: May 22, 2025

(30) Foreign Application Priority Data
Feb. 1, 2023 (EP) 23154389.3
Feb. 15, 2023 (LU) LU103072

(51) Int. Cl. A61K 31/454; A61K 31/4164; A61K 31/4192; A61K 31/4196; C07B 59/00; C07D 233/58; C07D 233/61; C07D 249/06; C07D 249/08

(52) U.S. Cl. CPC A61K 31/454; A61K 31/4164; A61K 31/4192; A61K 31/4196; C07B 59/002; C07D 233/58; C07D 233/61; C07D 249/06; C07D 249/08; C07B 2200/05

(57) ABSTRACT

The invention relates to the therapeutic application of multifunctional compounds that act as α₂ adrenoreceptor antagonists and cholinesterase inhibitors for the treatment of Alzheimer's disease and other neurodegenerative diseases, or the concomitant use of a mixture of an α₂ adrenoreceptor antagonist and a cholinesterase inhibitor to achieve the same effect.

### TECHNICAL FIELD

The present invention relates to the fields of medicinal chemistry and medicine, and compounds that have a dual mode of action, i.e., act as α₂ adrenoreceptor antagonists and cholinesterase inhibitors, as disease-modifying treatment of Alzheimer's disease and other neurodegenerative diseases. These compounds can be in the form of a mixture of enantiomers or in the form of pure enantiomers, in the form of pharmaceutically acceptable salts, and as hydrates or solvates thereof.

### BACKGROUND ART

Dementia is a grievous neurological condition that severely debilitates the patient's quality of life. By some estimates, over 50 million people in the world are beset by Alzheimer's disease (AD) (World Alzheimer Report 2022. 2022.). Neurotransmitter acetylcholine (ACh) plays an essential role in the formation and retrieval of memories, judgement, and orientation. In AD, cholinergic regions, e.g., Meynert's nucleus basalis and its projections to the frontal cortex, undergo significant neurodegradation and loss of cholinergic signalling (Huang, L.-K.; Chao, S.-P.; Hu, C.-J. J. Biomed. Sci. 2020, 27 (1), 18; Wang, H.; Zhang, H. ACS Chem. Neurosci. 2019, 10 (2), 852-862; Scarpini, E.; Schelterns, P.; Feldman, H. Lancet Neurol. 2003, 2 (9), 539-547). This, combined with cognitive improvement after applying cholinergic agonists or cholinesterase (ChE) inhibitors, gives evidence to the so-called cholinergic hypothesis of AD etiology (Contestabile, A. Behav. Brain Res. 2011, 221 (2), 334-340). Acetyl- (AChE) and butyrylcholinesterase (BChE), terminate cholinergic signalling by hydrolysing ACh. AChE is the main ChE that performs 80% of hydrolytic activity in the brain, being attached to the postsynaptic membrane. BChE, located at the glial cells, can function as an auxiliary ChE, taking over at high ACh concentrations when substrate inhibition of AChE takes place (Greig, N. H.; Utsuki, T.; Yu, Q.; Zhu, X.; Holloway, H. W.; Perry, T.; Lee, B.; Ingram, D. K.; Lahiri, D. K. Curr. Med. Res. Opin. 2001, 17 (3), 159-165; Lockridge, O. Pharmacol. Ther. 2015, 148, 34-46; Xing, S.; Li, Q.; Xiong, B.; Chen, Y.; Feng, F.; Liu, W.; Sun, H. Med. Res. Rev. 2021, 41 (2), 858-901). As AD progresses, not only cholinergic transmission but also the AChE activity subsides. To compensate, BChE takes over and terminates even up to 90% of the cholinergic signalling in the brain (Greig, N. H.; Utsuki, T.; Yu, Q.; Zhu, X.; Holloway, H. W.; Perry, T.; Lee, B.; Ingram, D. K.; Lahiri, D. K. Curr. Med. Res. Opin. 2001, 17 (3), 159-165; Campanari, M.-L.; García-Ayllón, M.-S.; Blazquez-Llorca, L.; Luk, W. K. W.; Tsim, K.; Sáez-Valero, J. J. Mol. Neurosci. 2014, 53 (3), 446-453). Additionally, both enzymes are deposited in senile plaques and AChE is implicated in Aβ aggregation through its peripheral aromatic binding site (Diamant, S.; Podoly, E.; Friedler, A.; Ligumsky, H.; Livnah, O.; Soreq, H. Proc. Natl. Acad. Sci. 2006, 103 (23), 8628-8633). The loss of cholinergic signalling not only exacerbates the cognitive status, but might also kindle the amyloid- and tau-induced microglial inflammation that the intact cholinergic pathways had previously held in check (Schmitz, T. W.; Soreq, H.; Poirier, J.; Spreng, R. N. J. Neurosci. 2020, 40 (9), 1931-1942). Selective BChE inhibitors in vivo ameliorated cognitive performance in aged rats or in scopolamine-treated mice (Cerbai, F.; Giovannini, M. G.; Melani, C.; Enz, A.; Pepeu, G. Eur. J. Pharmacol. 2007, 572 (2), 142-150; Košak, U.; Brus, B.; Knez, D.; Šink, R.; Zakelj, S.; Trontelj, J.; Pišlar, A.; Šlenc, J.; Gobec, M.; Živin, M.; Tratnjek, L.; Perše, M.; Salat, K.; Podkowa, A.; Filipek, B.; Nachon, F.; Brazzolotto, X.; Więckowska, A.; Malawska, B.; Stojan, J.; Raščan, I. M.; Kos, J.; Coquelle, N.; Colletier, J.-P.; Gobec, S. Sci. Rep. 2016, 6, 39495). Additionally, due to the selective inhibition of BChE over AChE, these compounds do not elicit peripheral parasympathomimetic side effects that are typical for AChE inhibitors.

However, cholinergic dysfunction is only one of many pathophysiological changes in AD, in addition to extracellular amyloid β (Aβ) plaques, dystrophic neurites with intracellular neurofibrillary tangles of aggregated protein tau, neuronal loss, amyloid angiopathy, oxidative damage, calcium imbalance, and neuroinflammation. The accumulation of neurofibrillary tangles, not amyloid plaques, positively correlates with AD severity (Masters, C. L.; Bateman, R.; Blennow, K.; Rowe, C. C.; Sperling, R. A.; Cummings, J. L. Nat. Rev. Dis. Primer 2015, 1, 15056; Du, X.; Wang, X.; Geng, M. Transl. Neurodegener. 2018, 7 (1), 2; Hampel, H.; Mesulam, M.-M.; Cuello, A. C.; Farlow, M. R.; Giacobini, E.; Grossberg, G. T.; Khachaturian, A. S.; Vergallo, A.; Cavedo, E.; Snyder, P. J.; Khachaturian, Z. S. Brain 2018, 141 (7), 1917-1933; Panza, F.; Lozupone, M.; Logroscino, G.; Imbimbo, B. P. Nat. Rev. Neurol. 2019, 15 (2), 73-88; Stefanovska, K.; Gajwani, M.; Tan, A. R. P.; Ahel, H. I.; Asih, P. R.; Volkerling, A.; Poljak, A.; Ittner. A. Sci. Adv. 2022, 8 (27), eab18809).

The brain noradrenergic system, located primarily in locus coeruleus, is critical for normal cognition and is affected at early stages of Alzheimer's disease. Furthermore, noradrenergic dysfunction is directly connected to the amyloid and tau pathologies. Zhang et al. have shown that extracellular oligomers of amyloid beta (Aβ) even in nanomolar concentrations allosterically bind to the third extracellular loop of α_{2A} adrenergic receptor (ADRA2A) and potentiate the norepinephrine-dependent activation of glycogen synthase kinase 3β (GSK3β) that leads to tau hyperphosphorylation, an effect prevented by applying α_{2A} adrenergic orthosteric antagonist (e.g., idazoxan) or lithium ions (GSK3β inhibition). This was also confirmed in two transgenic mice amyloid models, where long term treatment with idazoxan reduced Aβ plaque load, GSK3β activation, microglial activation and neuroinflammation, tau hyperphosphorylation, and ameliorated cognitive deficits in cognitively impaired mice with significant amyloid burden present in two behavioural assays. The authors attributed these disease-modifying effects to reduction in GSK3β activity and regulation of sorting-related receptor with A repeats (SorLA)-mediated APP trafficking. Additionally, a review of available AD patient cases from NACC database suggested that use of clonidine (α₂ agonist) further impaired cognitive function in dementia patients (Zhang, F.; Gannon, M.; Chen, Y.; Yan, S.; Zhang, S.; Feng, W.; Tao, J.; Sha, B.; Liu, Z.; Saito, T.; Saido, T.; Keene, C. D.; Jiao, K.; Roberson, E. D.; Xu, H.; Wang, Q. Sci. Transl. Med. 2020, 12 (526), eaay6931; Chen, Y.; Peng, Y.; Che, P.; Gannon, M.; Liu, Y.; Li, L.; Bu, G.; van Groen, T.; Jiao, K.; Wang, Q. Proc. Natl. Acad. Sci. 2014, 111 (48), 17296-17301; Gannon, M.; Che, P.; Chen, Y.; Jiao, K.; Roberson, E. D.; Wang, Q. Front. Neurosci. 2015, 9, 220).

4-(2-ethylindan-2-yl)-1H-imidazole (INN name: atipamezole) is a highly potent α₂ adrenergic antagonist with higher selectivity over ai adrenergic receptors than idazoxan. As a hydrochloride it is registered in veterinary medicine for reversing the sedative and analgesic effects of dexmedetomidine and medetomidine. Other atipamezole derivatives were also described in literature (Karjalainen, A. J.; Virtanen, R. E.; Karjalainen, A. L.; Kurkela, K. O. A. U.S. Pat. No. 4,689,339A, Aug. 25, 1987; Karjalainen, A. L.; Karjalainen, A. J. U.S. Pat. No. 5,026,868A, Jun. 25, 1991; Lammintausta, R. A. S.; Virtanen, R. E.; Riekkinen, P. J.; Riekkinen, J. P.; Sirvioe, J. S. I.; Miettinen, R. A.; Valjakka, A.; Airaksinen, M. M.; Nieminen, S. A.; Macdonald, E. GB2244431A, Dec. 4, 1991; Ratilainen, J.; Huhtala, P.; Karjalainen, A.; Karjalainen, A.; Haapalinna, A.; Virtanen, R.; Lehtimaki, J. WO2001085698A1, Nov. 15, 2001; Huhtala, P.; Karjalainen, A.; Haapalinna, A.; Lehtimäki, J.; Karjalainen, A.; Virtanen, R. U.S. Pat. No. 6,388,090B2, May 14, 2002. Ratilainen, J.; Huhtala, P.; Karjalainen, A.; Karjalainen, A.; Haapalinna, A.; Virtanen, R.; Lehtimaeki, J. AT302757T, Sep. 15, 2005; Pertovaara, A.; Haapalinna, A.; Sirviö, J.; Virtanen, R. CNS Drug Reviews 2005, 11 (3), 273-288; Vacher, B.; Bonnaud, B.; Marien, M.; Pauwels, P. US2006041001A1, Feb. 23, 2006; Vacher, B.; Funes, P.; Chopin, P.; Cussac, D.; Heusler, P.; Tourette, A.; Marien, M., J. Med. Chem. 2010, 53 (19), 6986-6995; Bonnaud, B.; Funes, P.; Jubault, N.; Vacher, B., European Journal of Organic Chemistry 2005, 2005 (15), 3360-3369.).

### SUMMARY OF INVENTION

The invention generally relates to multifunctional compounds that act as α₂ adrenoreceptor antagonists and cholinesterase inhibitors and to their therapeutic application in the treatment of Alzheimer's disease and other neurodegenerative diseases.

Specifically, the present inventors have discovered new multifunctional compounds that achieve both α₂ adrenoreceptor antagonism and cholinesterase inhibition. The compounds liberate adrenergic antagonist atipamezole (or its analogues) upon metabolic activation, which itself produces pseudo-irreversibly inhibited cholinesterase(s). Therefore, they can be classified as pleiotropic prodrugs, achieving dual effect—one before/during the metabolic activation, and one after the activation—in contrast with classical prodrugs which by definition exhibit little to no pharmacological activity before the activation (Toublet, F.-X.; Lalut, J.; Hatat, B.; Lecoutey, C.; Davis, A.; Since, M.; Corvaisier, S.; Freret, T.; Sopková-de Oliveira Santos, J.; Claeysen, S.; Boulouard, M.; Dallemagne, P.; Rochais, C. European Journal of Medicinal Chemistry 2021, 210, 113059; Toublet, F.-X.; Lecoutey, C.; Lalut, J.; Hatat, B.; Davis, A.; Since, M.; Corvaisier, S.; Freret, T.; Sopkova de Oliveira Santos, J.; Claeysen, S.; Boulouard, M.; Dallemagne, P.; Rochais, C. Molecules 2019, 24 (15), 2786; Wermuth, C. G.; Ganellin, C. R.; Lindberg, P.; Mitscher, L. A. Pure and Applied Chemistry 1998, 70 (5), 1129-1143.) The activation can take place in plasma, leaving the adrenergic moiety free to permeate to the blood-brain barrier to act on the α_{2A} adrenergic receptor in the central nervous system (CNS), leading to the first therapeutic benefit. Alternatively, the activation can take place with BChE present in the CNS, therefore also achieving the second therapeutic benefit, inhibition of brain BChE (Scheme I).

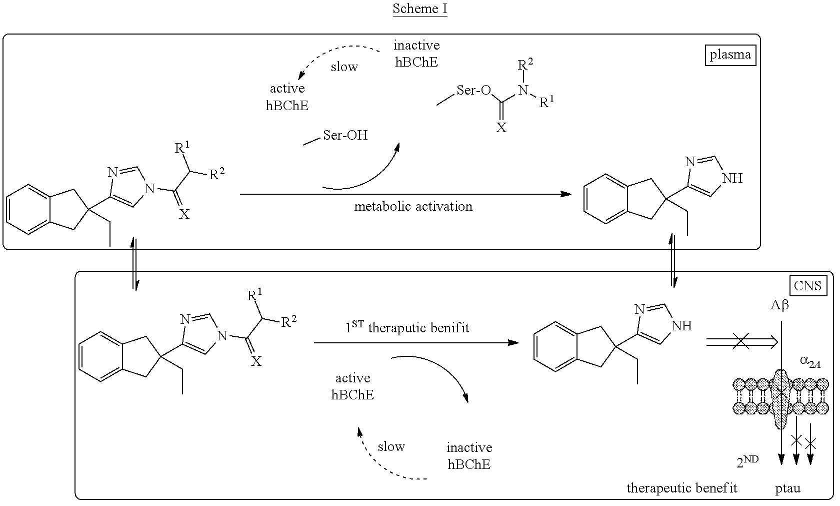

In a first aspect, the present invention relates to compounds of formula I and their pharmaceutically acceptable salts, racemate, diastereomer, enantiomer, regioisomer, hydrate or solvate thereof,

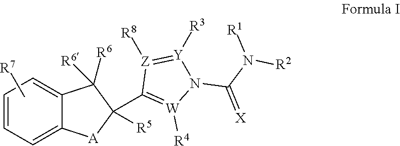

with R¹, R², R³, R⁴, R⁵, R⁶, R^{6′}, R⁷, R⁸, X, Y, W, Z, and A defined herein.

In a further aspect the present invention provides a pharmaceutical composition comprising a compound of the present invention, or a pharmaceutically acceptable salt thereof, and a pharmaceutically acceptable carrier, adjuvant, or vehicle.

In a further aspect the present invention provides a compound or pharmaceutical composition of the present invention for use in medicine.

In a further aspect the present invention provides a compound or pharmaceutical composition of the present invention for use in the treatment of a neurodegenerative disease, such as Alzheimer's disease, Parkinson's disease, and amyotrophic lateral sclerosis.

In a further aspect the present invention provides a synthetic route for 4-(2-ethylindan-2-yl)-1H-imidazole (INN name: atipamezole) that was not previously described. The enolate of an alkyl ester of indane-2-carboxylic acid is C-alkylated with iodoethane or bromoethane at low temperatures to afford alkyl 2-ethylindane-2-carboxylate. The ester is then hydrolyzed to the 2-ethylindane-2-carboxylic acid. A Weinreb amide is prepared from 2-ethylindane-2-carboxylic acid chloride, reduced to the corresponding aldehyde, which is then converted to 4-(2-ethylindan-2-yl)-1H-imidazole in a two-step van Leusen reaction.

The present invention may be summarized by the following items:
1. A compound of formula I

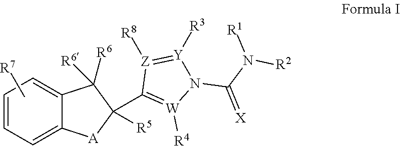

wherein
R¹ and R² are independently from each other a substituted or unsubstituted C₁-C₁₀ alkyl, substituted or unsubstituted C₂-C₁₀ alkenyl, substituted or unsubstituted C₂-C₁₀ alkynyl, substituted or unsubstituted C₃-C₁₀ cycloalkyl, substituted or unsubstituted five- to ten-membered aryl, or substituted or unsubstituted C₆-C₁₆ aralkyl; or R¹ and R² form together with the nitrogen atom to which they are attached a substituted or unsubstituted three- to ten-membered heterocycle;
R³ and R⁴ are independently from each other —H, -D, C₁-C₁₀ alkyl, substituted or unsubstituted C₂-C₁₀ alkenyl, substituted or unsubstituted C₂-C₁₀ alkynyl, substituted or unsubstituted C₃-C₁₀ cycloalkyl, substituted or unsubstituted five- to ten-membered aryl, or substituted or unsubstituted C₆-C₁₆ aralkyl, halogen, —NO₂, —CF₃, —OR or —SR, where R denotes a substituted or unsubstituted C₁-C₁₀ alkyl, substituted or unsubstituted C₂-C₁₀ alkenyl, substituted or unsubstituted C₂-C₁₀ alkynyl, substituted or unsubstituted C₃-C₁₀ cycloalkyl, substituted or unsubstituted five- to ten-membered aryl, or substituted or unsubstituted C₆-C₁₆ aralkyl;
R⁵ is a substituted or unsubstituted C₁-C₁₀ alkyl, substituted or unsubstituted C₂-C₁₀ alkenyl, substituted or unsubstituted C₂-C₁₀ alkynyl, substituted or unsubstituted C₃-C₁₀ cycloalkyl, substituted or unsubstituted five- to ten-membered aryl, substituted or unsubstituted C₆-C₁₆ aralkyl, —OR or —SR, where R denotes a substituted or unsubstituted C₁-C₁₀ alkyl, substituted or unsubstituted C₂-C₁₀ alkenyl, substituted or unsubstituted C₂-C₁₀ alkynyl, substituted or unsubstituted C₃-C₁₀ cycloalkyl, substituted or unsubstituted five- to 10-membered aryl, or substituted or unsubstituted C₆-C₁₆ aralkyl;
R⁶ and R^{6′} are independently from each other —H, -D, halogen, hydroxy, substituted or unsubstituted C₁-C₁₀ alkyl, substituted or unsubstituted C₂-C₁₀ alkenyl, substituted or unsubstituted C₂-C₁₀ alkynyl, substituted or unsubstituted C₃-C₁₀ cycloalkyl, substituted or unsubstituted five- to ten-membered aryl, substituted or unsubstituted C₆-C₁₆ aralkyl, —OR or —SR, where R denotes a substituted or unsubstituted C₁-C₁₀ alkyl, substituted or unsubstituted C₂-C₁₀ alkenyl, substituted or unsubstituted C₂-C₁₀ alkynyl, substituted or unsubstituted C₃-C₁₀ cycloalkyl, substituted or unsubstituted five- to ten-membered aryl, or substituted or unsubstituted C₆-C₁₆ aralkyl;
or, alternatively, R⁶ can form together with R⁵ or R^{6′} a substituted or unsubstituted C₃-C₆ cycloalkyl ring;
R⁷ represents one to four substituents on the aryl ring which are independently from each other selected from —H, -D, halogen, —NO₂, substituted or unsubstituted C₁-C₁₀ alkyl, substituted or unsubstituted C₂-C₁₀ alkenyl, substituted or unsubstituted C₂-C₁₀ alkynyl, substituted or unsubstituted C₃-C₁₀ cycloalkyl, substituted or unsubstituted five- to ten-membered aryl, substituted or unsubstituted C₆-C₁₆ aralkyl, —OR and —SR, where R denotes a substituted or unsubstituted C₁-C₁₀ alkyl, substituted or unsubstituted C₂-C₁₀ alkenyl, substituted or unsubstituted C₂-C₁₀ alkynyl, substituted or unsubstituted C₃-C₁₀ cycloalkyl, substituted or unsubstituted five- to ten-membered aryl, or substituted or unsubstituted C₆-C₁₆ aralkyl;
X is O, S, or Se;
Y, Z, and W are independently from each other carbon, nitrogen, sulfur, or oxygen;
R⁸ is —H, -D, halogen, —NO₂, substituted or unsubstituted C₁-C₆ alkyl, substituted or unsubstituted C₂-C₆ alkenyl, substituted or unsubstituted C₂-C₆ alkynyl, substituted or unsubstituted C₃-C₈ cycloalkyl, substituted or unsubstituted five- to ten-membered aryl, substituted or unsubstituted C₆-C₁₆ aralkyl, —OR and —SR, where R denotes a substituted or unsubstituted C₁-C₆ alkyl, substituted or unsubstituted C₂-C₆ alkenyl, substituted or unsubstituted C₂-C₆ alkynyl, substituted or unsubstituted C₃-C₈ cycloalkyl, substituted or unsubstituted five- to ten-membered aryl, or substituted or unsubstituted C₆-C₁₆ aralkyl; and
A is —CH₂—, —CH₂CH₂—, —O—, —CH(—OH)—, —C(═O)—, —CHR—, —CR₂— —CH(—OR)—, —S—, —SO—, —SO₂—, or —N(R)—, where R denotes a substituted or unsubstituted C₁-C₆ alkyl, substituted or unsubstituted C₂-C₆ alkenyl, substituted or unsubstituted C₂-C₆ alkynyl, substituted or unsubstituted C₃-C₆ cycloalkyl, substituted or unsubstituted five- to ten-membered aryl, or substituted or unsubstituted C₆-C₁₆ aralkyl;
or a pharmaceutically acceptable salt, racemate, diastereomer, enantiomer, regioisomer, hydrate or solvate thereof.
2. Compound according to item 1, wherein R¹ is a substituted or unsubstituted C₁-C₆ alkyl, substituted or unsubstituted C₂-C₆ alkenyl, substituted or unsubstituted C₂-C₆ alkynyl, substituted or unsubstituted C₃-C₈ cycloalkyl, substituted or unsubstituted five- to ten-membered aryl, or substituted or unsubstituted C₆-C₁₆ aralkyl.
3. Compound according to item 1, wherein R¹ is -Me, -Et, vinyl, ethynyl, —Pr, -iPr, —Bu, benzyl or phenethyl.
4. Compound according to item 1, wherein R¹ is a substituted or unsubstituted C₁-C₆ alkyl.
5. Compound according to item 1, wherein R¹ is a substituted or unsubstituted C₁-C₄ alkyl.
6. Compound according to item 1, wherein R¹ is -Me.
7. Compound according to item 1, wherein R¹ is -Et.
8. Compound according to any one of items 1 to 7, wherein R² is a substituted or unsubstituted C₁-C₆ alkyl, substituted or unsubstituted C₂-C₆ alkenyl, substituted or unsubstituted C₂-C₆ alkynyl, substituted or unsubstituted C₃-C₈ cycloalkyl, substituted or unsubstituted five- to ten-membered aryl, or substituted or unsubstituted C₆-C₁₆ aralkyl.
9. Compound according to any one of items 1 to 7, wherein R² is -Me, -Et, vinyl, ethynyl, —Pr, -iPr, —Bu, benzyl or phenethyl.
10. Compound according to any one of items 1 to 7, wherein R² is a substituted or unsubstituted C₁-C₆ alkyl.
11. Compound according to any one of items 1 to 7, wherein R² is a substituted or unsubstituted C₁-C₄ alkyl.
13. Compound according to any one of items 1 to 7, wherein R^{z} is -Me.
14. Compound according to any one of items 1 to 7, wherein R² is -Et.
15. Compound according to any one of items 1 to 7, wherein R¹ and R² are each -Me.
16. Compound according to any one of items 1 to 7, wherein R¹ and R² are each -Et.
17. Compound according to item 1, wherein R¹ and R² form together with the nitrogen atom to which they are attached a substituted or unsubstituted three- to ten-membered heterocycle.
18. Compound according to item 1, wherein or R¹ and R² form together with the nitrogen atom to which they are attached a substituted or unsubstituted five- or six-membered heterocycle, preferably a substituted or unsubstituted six-membered heterocycle.
19. Compound according to any one of items 1 to 18, wherein X is O or S, preferably X is O.
20. Compound according to any one of items 1 to 19, wherein Y is C.
21. Compound according to any one of items 1 to 19, wherein Y is N.
22. Compound according to any one of items 1 to 21, wherein Z is N.
23. Compound according to any one of items 1 to 21, wherein Z is C.
24. Compound according to any one of items 1 to 23, wherein R⁸ is —H.
25. Compound according to any one of items 1 to 24, wherein W is C or N.
26. Compound according to any one of items 1 to 24, wherein W is C.
27. Compound according to any one of items 1 to 24, wherein W is N.
28. Compound according to any one of items 1 to 27, wherein A is —CH₂—, —CH₂CH₂—, —O—, —CH(—OH)—, —C(═O)—, —CHR—, —CR₂— —CH(—OR)—, —S—, —SO—, —SO₂—, or —N(R)—, where R denotes a substituted or unsubstituted C₁-C₄ alkyl, substituted or unsubstituted C₂-C₄ alkenyl, substituted or unsubstituted C₂-C₄ alkynyl, substituted or unsubstituted C₃-C₆ cycloalkyl, substituted or unsubstituted five- or six-membered aryl, or substituted or unsubstituted C₆-C₁₀ aralkyl.
29. Compound according to any one of items 1 to 27, wherein A is —CH₂—.
30. Compound according to any one of items 1 to 29, wherein R⁶ is —H, -D, halogen, substituted or unsubstituted C₁-C₆ alkyl, substituted or unsubstituted C₂-C₆ alkenyl, substituted or unsubstituted C₂-C₆ alkynyl, substituted or unsubstituted C₃-C₈ cycloalkyl, substituted or unsubstituted five- to ten-membered aryl, or substituted or unsubstituted C₆-C₁₆ aralkyl —OR or —SR, where R denotes a substituted or unsubstituted C₁-C₆ alkyl, substituted or unsubstituted C₂-C₆ alkenyl, substituted or unsubstituted C₂-C₆ alkynyl, substituted or unsubstituted C₃-C₈ cycloalkyl, substituted or unsubstituted five- to ten-membered aryl, or substituted or unsubstituted C₆-C₁₆ aralkyl; and/or
   wherein R^{6′} is —H, -D, halogen, substituted or unsubstituted C₁-C₆ alkyl, substituted or unsubstituted C₂-C₆ alkenyl, substituted or unsubstituted C₂-C₆ alkynyl, substituted or unsubstituted C₃-C₈ cycloalkyl, substituted or unsubstituted five- to ten-membered aryl, or substituted or unsubstituted C₆-C₁₆ aralkyl, —OR or —SR, where R denotes a substituted or unsubstituted C₁-C₆ alkyl, substituted or unsubstituted C₂-C₆ alkenyl, substituted or unsubstituted C₂-C₆ alkynyl, substituted or unsubstituted C₃-C₈ cycloalkyl, substituted or unsubstituted five- to ten-membered aryl, or substituted or unsubstituted C₆-C₁₆ aralkyl.
31. Compound according to any one of items 1 to 30, wherein R⁶ is —H and/or wherein R^{6′} is —H.
32. Compound according to any one of items 1 to 18, which is a compound of Formula II

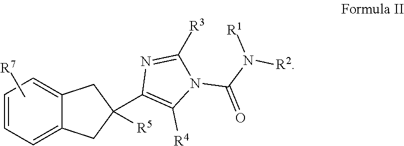

33. Compound according to any one of items 1 to 18, which is a compound of Formula III

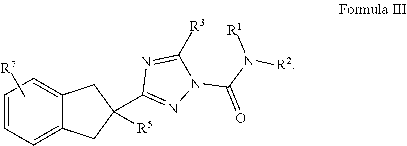

34. Compound according to any one of items 1 to 18, which is a compound of Formula IV

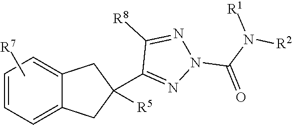

35. Compound according to item 34, wherein R¹ is —H.
36. Compound according to any one of items 1 to 33, wherein R³ is —H, -D, substituted or unsubstituted C₁-C₄ alkyl, substituted or unsubstituted C₂-C₄ alkenyl, substituted or unsubstituted C₂-C₄ alkynyl, substituted or unsubstituted C₃-C₆ cycloalkyl, substituted or unsubstituted five- or six-membered aryl, or substituted or unsubstituted C₆-C₁₀ aralkyl, halogen, —NO₂, —CF₃, —OR or —SR, where R denotes a substituted or unsubstituted C₁-C₄ alkyl, substituted or unsubstituted C₂-C₄ alkenyl, substituted or unsubstituted C₂-C₄ alkynyl, substituted or unsubstituted C₃-C₆ cycloalkyl, substituted or unsubstituted five- or six-membered aryl, or substituted or unsubstituted C₆-C₁₀ aralkyl.
37. Compound according to any one of items 1 to 33, wherein R³ is —H.
38. Compound according to any one of items 1 to 32, wherein R⁴ is —H, -D, substituted or unsubstituted C₁-C₄ alkyl, substituted or unsubstituted C₂-C₄ alkenyl, substituted or unsubstituted C₂-C₄ alkynyl, substituted or unsubstituted C₃-C₆ cycloalkyl, substituted or unsubstituted five- or six-membered aryl, or substituted or unsubstituted C₆-C₁₀ aralkyl, halogen, —NO₂, —CF₃, —OR or —SR, where R denotes a substituted or unsubstituted C₁-C₄ alkyl, substituted or unsubstituted C₂-C₄ alkenyl, substituted or unsubstituted C₂-C₄ alkynyl, substituted or unsubstituted C₃-C₆ cycloalkyl, substituted or unsubstituted five- or six-membered aryl, or substituted or unsubstituted C₆-C₁₀ aralkyl.
39. Compound according to any one of items 1 to 32, wherein R⁴ is —H.
40. Compound according to any one of items 1 to 39, wherein R⁵ is a substituted or unsubstituted C₁-C₆ alkyl, substituted or unsubstituted C₂-C₆ alkenyl, substituted or unsubstituted C₂-C₆ alkynyl, substituted or unsubstituted C₃-C₈ cycloalkyl, substituted or unsubstituted five- to ten-membered aryl, substituted or unsubstituted C₆-C₁₆ aralkyl, —OR or —SR, where R denotes a substituted or unsubstituted C₁-C₆ alkyl, substituted or unsubstituted C₂-C₆ alkenyl, substituted or unsubstituted C₂-C₆ alkynyl, substituted or unsubstituted C₃-C₈ cycloalkyl, substituted or unsubstituted five- to ten-membered aryl, or substituted or unsubstituted C₆-C₁₆ aralkyl.
41. Compound according to any one of items 1 to 39, wherein R⁵ is -Me, -Et, vinyl, ethynyl, —Pr, -iPr, -Bu, benzyl or phenethyl.
42. Compound according to any one of items 1 to 39, wherein R⁵ is -Me.
43. Compound according to any one of items 1 to 39, wherein R⁵ is -Et.
44. Compound according to any one of items 1 to 43, wherein R⁷ represents one to four substituents on the aryl ring which are independently from each other selected from —H, -D, halogen, —NO₂, substituted or unsubstituted C₁-C₆ alkyl, substituted or unsubstituted C₂-C₆ alkenyl, substituted or unsubstituted C₂-C₆ alkynyl, substituted or unsubstituted C₃-C₈ cycloalkyl, substituted or unsubstituted five- to ten-membered aryl, or substituted or unsubstituted C₆-C₁₆ aralkyl, —OR and —SR groups, where R denotes a substituted or unsubstituted C₁-C₆ alkyl, substituted or unsubstituted C₂-C₆ alkenyl, substituted or unsubstituted C₂-C₆ alkynyl, substituted or unsubstituted C₃-C₈ cycloalkyl, substituted or unsubstituted five- to ten-membered aryl, or substituted or unsubstituted C₆-C₁₆ aralkyl.
45. Compound according to any one of items 1 to 44, wherein R⁷ represents one to four substituents on the aryl ring which are independently from each other selected from —H, -Me, -Et, vinyl, ethynyl, —Pr, -iPr, -Bu, benzyl and phenethyl.
46. Compound according to any one of items 1 to 44, wherein R⁷ is —H.
47. Compound according to any one of items 1 to 18, wherein R³ is —H, R⁴ is —H, R⁵ is -Et, R⁶ is —H, R⁷ is —H, and R⁸ is —H.
48. Compound according to item 47, wherein X is O and A is —CH₂—.
49. Compound according to item 47 or 48, wherein Y is C, Z is N, and W is C.
50. Compound according to item 1, which is selected from the group consisting of:
N-ethyl-4-(2-ethyl-2,3-dihydro-1H-inden-2-yl)-N-methyl-1H-imidazole-1-carboxamide;
4-(2-ethyl-2,3-dihydro-1H-inden-2-yl)-N,N-dimethyl-1H-imidazole-1-carboxamide;
N,N-diethyl-4-(2-ethyl-2,3-dihydro-1H-inden-2-yl)-1H-imidazole-1-carboxamide;
1-(dimethylcarbamoyl)-4-(2-ethylindan-2-yl)-3-methyl-1H-imidazol-3-ium trifluoromethanesulfonate;
4-(2-ethylindan-2-yl)-N,N-dimethyl-2H-1,2,3-triazole-2-carboxamide;
3-(2-ethylindan-2-yl)-N,N-dimethyl-1H-1,2,4-triazole-1-carboxamide;
N-benzyl-4-(2-ethylindan-2-yl)-N-methyl-1H-imidazole-1-carboxamide;
(4-(2-ethylindan-2-yl)-1H-imidazol-1-yl)(piperidin-1-yl)methanone;
4-(2-ethylindan-2-yl)-N,N-dimethyl-1H-imidazole-1-carbothioamide; and
N,N-diethyl-4-(2-(ethyl-d₅)indan-2-yl)-1H-imidazole-1-carboxamide.
   51. Compound according to item 1, which is selected from the group consisting of:
N-ethyl-4-(2-ethyl-2,3-dihydro-1H-inden-2-yl)-N-methyl-1H-imidazole-1-carboxamide;
4-(2-ethyl-2,3-dihydro-1H-inden-2-yl)-N,N-dimethyl-1H-imidazole-1-carboxamide; and
N,N-diethyl-4-(2-ethyl-2,3-dihydro-1H-inden-2-yl)-1H-imidazole-1-carboxamide.
   52. Compound according to item 1, which is a compound of Formula V

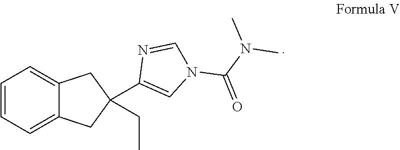 [SMILES: CCC1(c2cn(C(=O)N(C)C)cn2)Cc2ccccc2C1]

53. Compound according to item 1, which is a compound of Formula VI

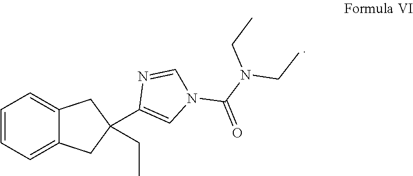 [SMILES: CCN(CC)C(=O)n1cnc(C2(CC)Cc3ccccc3C2)c1]

54. Pharmaceutical composition comprising a compound according to any one of items 1 to 53, or a pharmaceutically acceptable salt thereof, and a pharmaceutically acceptable carrier, adjuvant, or vehicle.
55. Compound according to any one of items 1 to 53 or pharmaceutical composition according to item 54 for use in medicine.
56. Compound according to any one of items 1 to 53 or pharmaceutical composition according to item 54 for use in for use in the treatment of a neurodegenerative disease.
57. Compound or pharmaceutical composition for use according to item 56, wherein the neurodegenerative disease is Alzheimer's disease, Parkinson's disease, or amyotrophic lateral sclerosis.
58. Process for the preparation of compounds according to any one of items 1 to 53, the process comprising reacting a compound of the formula VII

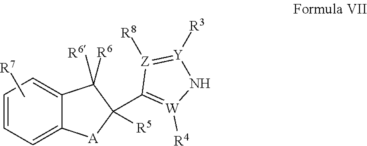

or a regioisomer thereof with a compound of formula VIII

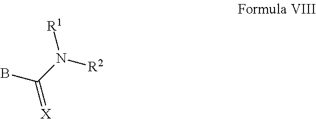

in the presence of a suitable base, such as an inorganic or organic base (e.g., triethylamine, DIPEA, pyridine, N-methylimidazole, LDA, potassium carbonate, sodium carbonate, sodium hydroxide, potassium hydroxide, potassium tert-butoxide, etc.);
wherein R¹, R², R³, R⁴, R⁵, R⁶, R^{6′}, R⁷, R⁸, X, Y, Z and W are as defined in any one of items 1 to 53, and wherein B represents a suitable leaving group, such as halogen (F, Cl, Br) or N-alkylimidazole.
59. Process for the preparation of 4-(2-ethylindan-2-yl)-1H-imidazole, which comprises reacting 2-ethylindane-2-carbaldehyde with arylsulfonylmethyl isocyanide (preferably toluenesulfonylmethyl isocyanide), followed by reaction with ammonia.
60. Process for the preparation of 2-ethylindane-2-carbaldehyde, which comprises reacting 2-ethyl-N-methoxy-N-methylindan-2-carboxamide with lithium aluminium hydride or diisobutylaluminium hydride.
61. Process for the preparation of 2-ethyl-N-methoxy-N-methylindan-2-carboxamide, which comprises reacting the acid chloride of 2-ethylindane-2-carboxylic acid with N,O-dimethylhydroxylamine hydrochloride in the presence of a base (e.g., triethylamine, DIPEA, pyridine, N-methylimidazole, potassium carbonate, sodium carbonate, sodium hydrogencarbonate, potassium tert-butoxide, etc.).

### DETAILED DESCRIPTION OF INVENTION

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety. In case of conflict, the present specification, including definitions, will control. In addition, the materials, methods, and examples are illustrative only and not intended to be limiting.

### Definitions

The term “alkyl” represents saturated, linear or branched hydrocarbons, which may be unsubstituted or substituted once or several times. “C₁-C₁₀ alkyl”, as referred to in the present invention, means an alkyl radical of 1, 2, 3, 4, 5, 6, 7, 8, 9 or 10 carbon atoms. Preferably, alkyl is C₁₋₆ alkyl like methyl, ethyl, propyl, n-propyl, isopropyl, butyl, n-butyl, tert-butyl, isobutyl, sec-butyl, pentyl or hexyl; and more preferably is C₁₋₄ alkyl like methyl, ethyl, propyl, n-propyl, isopropyl, butyl, n-butyl, tert-butyl, isobutyl or sec-butyl.

The term “alkenyl” represents a linear or branched hydrocarbon moiety which has one or more double bonds in the chain, and which may be unsubstituted or substituted once or several times. It encompasses groups like e.g. CH═CH═CH3. “C₂-C₁₀ alkenyl”, as referred to in the present invention, means an alkenyl radical of 1, 2, 3, 4, 5, 6, 7, 8, 9 or 10 carbon atoms. Preferably, alkenyl is C₂₋₆ alkenyl like ethylene, vinyl (ethenyl), propylene, allyl (2-propenyl), butylene, pentylene, or hexylene, and more preferably alkenyl is C₂₋₄ alkenyl like ethylene, vinyl (ethenyl), propylene, allyl (2-propenyl), or butylene.

The term “alkynyl” represent a linear or branched hydrocarbon moiety which has one or more triple bonds in the chain, and which may be unsubstituted or substituted once or several times. It encompasses groups like e.g. —C≡C—CH3 (1-propinyl). “C₂-C₁₀ alkynyl”, as referred to in the present invention, means an alkynyl radical of 1, 2, 3, 4, 5, 6, 7, 8, 9 or 10 carbon atoms.

Preferably, alkynyl is C₂₋₆ alkynyl like ethyne, propyne, butyene, pentyne, or hexyne; and more preferably alkynyl is C₂₋₄ alkynyl like ethyne, propyne, butyene, pentyne, or hexyne.

The term “cycloalkyl” represents saturated and unsaturated (but not aromatic) cyclic hydrocarbons (without a heteroatom in the ring), which can be unsubstituted or once or several times substituted. “C₃-C₁₀ cycloalkyl”, as referred to in the present invention, means an cycloalkyl of 3, 4, 5, 6, 7, 8, 9 or 10 carbon atoms. Examples are cyclopropyl, 2-methylcyclopropyl, cyclopropylmethyl, cyclobutyl, cyclopentyl, cyclopentylmethyl, cyclohexyl, cycloheptyl, cyclooctyl, and also adamantly. Preferably, cycloalkyl is C₃₋₇ cycloalkyl like cyclopropyl, cyclobutyl, cyclopentyl, cyclohexyl, or cycloheptyl or is C₃₋₆ cycloalkyl like cyclopropyl, cyclobutyl, cyclopentyl or cyclohexyl, especially cyclopentyl or cyclohexyl.

The term “aryl” represents a ring system with at least one aromatic ring but without heteroatoms even in only one of the rings, which can be unsubstituted or once or several times substituted. “five- to ten-membered aryl”, as referred to in the present invention, means an aryl of 5, 6, 7, 8, 9 or 10 carbon atoms. Particularly aryl is a monocyclic ring containing 5 or 6 atoms or a bicyclic ring containing 9 or 10 atoms. Examples are phenyl and naphthyl. Most preferably, aryl is phenyl.

The term “aralkyl” represents an aryl group attached to the adjacent atom by an alkyl, alkenyl or alkynyl. Like the aryl groups, where indicated, the aralkyl groups can also be optionally substituted. “C₆-C₁₆ aralkyl”, as referred to in the present invention, means an aralkyl of 6, 7, 8, 9, 10, 11, 12, 13, 14, 15 or 16 carbon atoms in total. Examples include but are not limited to benzyl, benzhydryl, trityl, phenethyl, 3-phenylpropyl, 2-phenylpropyl, 4-phenylbutyl and naphthylmethyl.

In a general definition a “heterocycle” represents a heterocyclic ring system ring system with at least one saturated or unsaturated ring which contains one or more heteroatoms from the group consisting of nitrogen, oxygen and/or sulfur in the ring. A heterocyclic group can also be substituted once or several times. A “heterocycle” can be aromatic or non-aromatic. “three- to ten-membered heterocycle”, as referred to in the present invention, means a heterocycle of 3, 4 5, 6, 7, 8, 9 or 10 atoms including the one or more heteroatoms. Examples include heteroaryls such as furan, benzofuran, thiophene, benzothiophene, pyrrole, pyridine, pyrimidine, pyrazine, quinoline, isoquinoline, phthalazine, benzo-1,2,5-thiadiazole, benzothiazole, indole, benzotriazole, benzodioxolane, benzodioxane, carbazole and quinazoline. Preferably, a “heterocycle” it is a heterocyclic ring system of one or two saturated or unsaturated rings (monocyclic or bicyclic ring system) of which at least one ring contains one or more heteroatoms from the group consisting of nitrogen, oxygen and/or sulfur in the ring. Preferred examples include imidazole, oxadiazole, tetrazole, pyridine, pyrimidine, piperidine, piperazine, benzofuran, benzimidazole, indazole, benzothiazole, benzodiazole, thiazole, benzothiazole, tetrahydropyrane, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole oxopyrrolidine, pyrimidine, benzodioxolane, benzodioxane, carbazole and quinazoline, especially is pyridine, pyrazine, indazole, benzodioxane, thiazole, benzothiazole, morpholine, tetrahydropyrane, pyrazole, imidazole, piperidine, pyrazole, thiophene, indole, benzimidazole, pyrrolo[2,3b]pyridine, benzoxazole, oxopyrrolidine, and pyrimidine.

The term “halogen” is used to denote fluoro, chloro, bromo, or iodo. Particularly, halogens are fluoro or chloro.

The term “independently” means that a substituent can be the same or a different definition for each item.

In general, the term “substituted”, whether preceded by the term “optionally” or not, refers to the replacement of hydrogen radicals on a carbon or nitrogen atom in a given structure with the radical of a specified substituent. Non-limiting examples of possible substituents include deuterium, halogen, hydroxy, keto groups, C₁-C₆ alkyloxy (such as methoxy or ethoxy), and C₁-C₆ alkylthio. Accordingly, if a certain group in a given R variable is indicated as being “substituted” (such as a “substituted alkyl in R¹) it may be substituted with at least one substituent selected from deuterium, halogen, hydroxy, keto groups, C₁-C₆ alkyloxy (such as methoxy or ethoxy), and C₁-C₆ alkylthio. Unless otherwise indicated, an optionally substituted group can have a substituent at each substitutable position of the group, and when more than one position in any given structure can be substituted with more than one substituent selected from a specified group, the substituent can be either the same or different at every position.

The term “pharmaceutically acceptable salt” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid and the like, in particular hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcysteine and the like. In addition these salts may be prepared by addition of an inorganic base or an organic base to the free acid. Salts derived from an inorganic base include, but are not limited to, the sodium, potassium, lithium, ammonium, calcium, magnesium salts and the like. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyimine resins and the like. Particular pharmaceutically acceptable salts of compounds of formula I are the hydrochloride salts, methanesulfonic acid salts, and citric acid salts.

The term “solvate” is to be understood as meaning any form of the active compound according to the invention in which this compound has attached to it via non-covalent binding another molecule (most likely a polar solvent). Especially preferred examples include hydrates and alcoholates, like methanolates or ethanolates.

Unless otherwise stated, the compounds of the invention are also meant to include compounds which differ only in the presence of one or more isotopically enriched atoms. For example, compounds having the present structures except for the replacement of hydrogen by a deuterium or tritium are within the scope of this invention.

### Compounds of the Invention

In its broadest aspect the present invention provides a compound of formula I

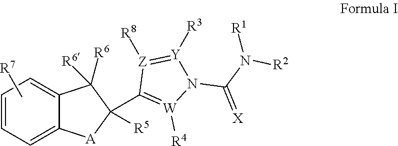

wherein
R¹ and R² are independently from each other a substituted or unsubstituted C₁-C₁₀ alkyl, substituted or unsubstituted C₂-C₁₀ alkenyl, substituted or unsubstituted C₂-C₁₀ alkynyl, substituted or unsubstituted C₃-C₁₀ cycloalkyl, substituted or unsubstituted 5-10 membered aryl, or substituted or unsubstituted C₆-C₁₆ aralkyl; or R¹ and R² form together with the nitrogen atom to which they are attached a substituted or unsubstituted three- to ten-membered heterocycle;
R³ and R⁴ are independently from each other —H, -D, C₁-C₁₀ alkyl, substituted or unsubstituted C₂-C₁₀ alkenyl, substituted or unsubstituted C₂-C₁₀ alkynyl, substituted or unsubstituted C₃-C₁₀ cycloalkyl, substituted or unsubstituted five- to ten-membered aryl, or substituted or unsubstituted C₆-C₁₆ aralkyl —NO₂, —CF₃, —OR or —SR, where R denotes a substituted or unsubstituted C₁-C₁₀ alkyl, substituted or unsubstituted C₂-C₁₀ alkenyl, substituted or unsubstituted C₂-C₁₀ alkynyl, substituted or unsubstituted C₃-C₁₀ cycloalkyl, substituted or unsubstituted five- to ten-membered aryl, or substituted or unsubstituted C₆-C₁₆ aralkyl;
R⁵ is a substituted or unsubstituted C₁-C₁₀ alkyl, substituted or unsubstituted C₂-C₁₀ alkenyl, substituted or unsubstituted C₂-C₁₀ alkynyl, substituted or unsubstituted C₃-C₁₀ cycloalkyl, substituted or unsubstituted five- to ten-membered aryl, substituted or unsubstituted C₆-C₁₆ aralkyl, —OR or —SR, where R denotes a substituted or unsubstituted C₁-C₁₀ alkyl, substituted or unsubstituted C₂-C₁₀ alkenyl, substituted or unsubstituted C₂-C₁₀ alkynyl, substituted or unsubstituted C₃-C₁₀ cycloalkyl, substituted or unsubstituted five- to ten-membered aryl, or substituted or unsubstituted C₆-C₁₆ aralkyl;
R⁶ and R^{6′} are independently from each other —H, -D, halogen, hydroxy, substituted or unsubstituted C₁-C₁₀ alkyl, substituted or unsubstituted C₂-C₁₀ alkenyl, substituted or unsubstituted C₂-C₁₀ alkynyl, substituted or unsubstituted C₃-C₁₀ cycloalkyl, substituted or unsubstituted five- to ten-membered aryl, substituted or unsubstituted C₆-C₁₆ aralkyl, —OR or —SR, where R denotes a substituted or unsubstituted C₁-C₁₀ alkyl, substituted or unsubstituted C₂-C₁₀ alkenyl, substituted or unsubstituted C₂-C₁₀ alkynyl, substituted or unsubstituted C₃-C₁₀ cycloalkyl, substituted or unsubstituted five- to ten-membered aryl aryl, or substituted or unsubstituted C₆-C₁₆ aralkyl;
or, alternatively, R⁶ can form together with R⁵ or R^{6′} a substituted or unsubstituted C₃-C₆ cycloalkyl ring;
R⁷ represents one to four substituents on the aryl ring which are independently from each other selected from —H, -D, halogen, —NO₂, substituted or unsubstituted C₁-C₁₀ alkyl, substituted or unsubstituted C₂-C₁₀ alkenyl, substituted or unsubstituted C₂-C₁₀ alkynyl, substituted or unsubstituted C₃-C₁₀ cycloalkyl, substituted or unsubstituted five- to ten-membered aryl, substituted or unsubstituted C₆-C₁₆ aralkyl, —OR and —SR, where R denotes a substituted or unsubstituted C₁-C₁₀ alkyl, substituted or unsubstituted C₂-C₁₀ alkenyl, substituted or unsubstituted C₂-C₁₀ alkynyl, substituted or unsubstituted C₃-C₁₀ cycloalkyl, substituted or unsubstituted five- to ten-membered aryl, or substituted or unsubstituted C₆-C₁₆ aralkyl;
X is O, S, or Se;
Y, Z, and W are independently from each other carbon, nitrogen, sulfur, or oxygen;
R⁸ is —H, -D, halogen, —NO₂, substituted or unsubstituted C₁-C₅ alkyl, substituted or unsubstituted C₁-C₅ alkenyl, substituted or unsubstituted C₁-C₅ alkynyl, substituted or unsubstituted C₃-C₈ cycloalkyl, substituted or unsubstituted five- to ten-membered aryl, substituted or unsubstituted C₆-C₁₆ aralkyl, —OR and —SR, where R denotes a substituted or unsubstituted C₁-C₆ alkyl, substituted or unsubstituted C₂-C₆ alkenyl, substituted or unsubstituted C₂-C₆ alkynyl, substituted or unsubstituted C₃-C₈ cycloalkyl, substituted or unsubstituted five- to ten-membered aryl, or substituted or unsubstituted C₆-C₁₆ aralkyl; and
A is —CH₂—, —CH₂CH₂—, —O—, —CH(—OH)—, —C(═O)—, —CHR—, —CR₂— —CH(—OR)—, —S—, —SO—, —SO₂—, or —N(R)—, where R denotes a substituted or unsubstituted C₁-C₆ alkyl, substituted or unsubstituted C₂-C₆ alkenyl, substituted or unsubstituted C₂-C₆ alkynyl, substituted or unsubstituted C₃-C₆ cycloalkyl, substituted or unsubstituted five- to ten-membered aryl, or substituted or unsubstituted C₆-C₁₆ aralkyl;
   or a pharmaceutically acceptable salt, racemate, diastereomer, enantiomer, regioisomer, hydrate or solvate thereof.

According to some embodiments, R¹ is a substituted or unsubstituted C₁-C₆ alkyl, substituted or unsubstituted C₂-C₆ alkenyl, substituted or unsubstituted C₂-C₆ alkynyl, substituted or unsubstituted C₃-C₈ cycloalkyl, substituted or unsubstituted five- to ten-membered aryl, or substituted or unsubstituted C₆-C₁₆ aralkyl.

According to some embodiments, R¹ is a substituted or unsubstituted C₁-C₄ alkyl, substituted or unsubstituted C₂-C₄ alkenyl, substituted or unsubstituted C₂-C₄ alkynyl, substituted or unsubstituted C₃-C₆ cycloalkyl, substituted or unsubstituted five- or six-membered aryl, or substituted or unsubstituted C₆-C₁₀ aralkyl.

According to some embodiments, R¹ is a substituted or unsubstituted C₁-C₆ alkyl, substituted or unsubstituted C₂-C₆ alkenyl, or substituted or unsubstituted C₂-C₆ alkynyl.

According to some embodiments, R¹ is a substituted or unsubstituted C₁-C₄ alkyl, substituted or unsubstituted C₂-C₄ alkenyl, or substituted or unsubstituted C₂-C₄ alkynyl.

According to some embodiments, R¹ is -Me, -Et, vinyl, ethynyl, —Pr, -iPr, -Bu, benzyl or phenethyl.

According to some embodiments, R¹ is a substituted or unsubstituted C₁-C₆ alkyl.

According to some embodiments, R¹ is a substituted or unsubstituted C₁-C₄ alkyl.

According to some embodiments, R¹ is -Me.

According to some embodiments, R¹ is -Et.

According to some embodiments, R² is a substituted or unsubstituted C₁-C₆ alkyl, substituted or unsubstituted C₂-C₆ alkenyl, substituted or unsubstituted C₂-C₆ alkynyl, substituted or unsubstituted C₃-C₈ cycloalkyl, substituted or unsubstituted five- to ten-membered aryl, or substituted or unsubstituted C₆-C₁₆ aralkyl.

According to some embodiments, R² is a substituted or unsubstituted C₁-C₄ alkyl, substituted or unsubstituted C₂-C₄ alkenyl, substituted or unsubstituted C₂-C₄ alkynyl, substituted or unsubstituted C₃-C₆ cycloalkyl, substituted or unsubstituted five- or six-membered aryl, or substituted or unsubstituted C₆-C₁₀ aralkyl.

According to some embodiments, R² is a substituted or unsubstituted C₁-C₆ alkyl, substituted or unsubstituted C₂-C₆ alkenyl, or substituted or unsubstituted C₂-C₆ alkynyl.

According to some embodiments, R² is a substituted or unsubstituted C₁-C₄ alkyl, substituted or unsubstituted C₂-C₄ alkenyl, or substituted or unsubstituted C₂-C₄ alkynyl.

According to some embodiments, R² is -Me, -Et, vinyl, ethynyl, —Pr, -iPr, -Bu, benzyl or phenethyl.

According to some embodiments, R² is a substituted or unsubstituted C₁-C₆ alkyl.

According to some embodiments, R² is a substituted or unsubstituted C₁-C₄ alkyl.

According to some embodiments, R² is -Me.

According to some embodiments, R² is -Et.

According to some embodiments, R¹ and R² are each -Me.

According to some embodiments, R¹ and R² are each -Et.

According to some embodiments, R¹ and R² form together with the nitrogen atom to which they are attached a substituted or unsubstituted three- to ten-membered heterocycle.

According to some embodiments, R¹ and R² form together with the nitrogen atom to which they are attached a substituted or unsubstituted five- or six-membered heterocycle.

According to some embodiments, R¹ and R² form together with the nitrogen atom to which they are attached a substituted or unsubstituted five-membered heterocycle.

According to some embodiments, R¹ and R² form together with the nitrogen atom to which they are attached a substituted or unsubstituted six-membered heterocycle.

According to some embodiments, R¹ and R² form together with the nitrogen atom to which they are attached a substituted or unsubstituted piperidine ring.

According to some embodiments, X is O or S.

According to some embodiments, X is O.

According to some embodiments, X is S.

According to some embodiments, Y is C or N.

According to some embodiments, Y is C.

According to some embodiments, Y is N.

According to some embodiments, Z is C or N.

According to some embodiments, Z is N.

According to some embodiments, Z is C.

According to some embodiments, R⁸ is —H, -D, halogen, —NO₂, substituted or unsubstituted C₁-C₄ alkyl, substituted or unsubstituted C₂-C₄ alkenyl, substituted or unsubstituted C₂-C₄ alkynyl, substituted or unsubstituted C₃-C₆ cycloalkyl, substituted or unsubstituted five- or six-membered aryl, substituted or unsubstituted C₆-C₁₀ aralkyl, —OR or —SR, where R denotes a substituted or unsubstituted C₁-C₄ alkyl, substituted or unsubstituted C₂-C₄ alkenyl, substituted or unsubstituted C₂-C₄ alkynyl, substituted or unsubstituted C₃-C₆ cycloalkyl, substituted or unsubstituted five- or six-membered aryl, or substituted or unsubstituted C₆-C₁₀ aralkyl.

According to some embodiments, R⁸ is —H, -D, halogen, —NO₂, substituted or unsubstituted C₁-C₄ alkyl, substituted or unsubstituted C₂-C₄ alkenyl, substituted or unsubstituted C₂-C₄ alkynyl, —OR or —SR, where R denotes a substituted or unsubstituted C₁-C₄ alkyl, substituted or unsubstituted C₂-C₄ alkenyl, substituted or unsubstituted C₂-C₄ alkynyl.

According to some embodiments, R⁸ is a substituted or unsubstituted C₁-C₄ alkyl.

According to some embodiments, R⁸ is —H.

According to some embodiments, W is C or N.

According to some embodiments, W is C.

According to some embodiments, W is N.

According to some embodiments, A is —CH₂—, —CH₂CH₂—, —O—, —CH(—OH)—, —C(═O)—, —CHR—, —CR₂— —CH(—OR)—, —S—, —SO—, —SO₂—, or —N(R)—, where R denotes a substituted or unsubstituted C₁-C₄ alkyl, substituted or unsubstituted C₂-C₄ alkenyl, substituted or unsubstituted C₂-C₄ alkynyl, substituted or unsubstituted C₃-C₆ cycloalkyl, substituted or unsubstituted 5-6 membered aryl, or substituted or unsubstituted C₆-C₁₀ aralkyl.

According to some embodiments, A is —CH₂—, —CH₂CH₂—, —O—, —CH(—OH)—, —C(═O)—, —CHR—, —CR₂— —CH(—OR)—, —S—, —SO—, —SO₂—, or —N(R)—, where R denotes a substituted or unsubstituted C₁-C₄ alkyl, substituted or unsubstituted C₂-C₄ alkenyl, substituted or unsubstituted C₂-C₄ alkynyl.

According to some embodiments, A is —CH₂—.

According to some embodiments, R⁶ is —H, -D, halogen, substituted or unsubstituted C₁-C₆ alkyl, substituted or unsubstituted C₂-C₆ alkenyl, substituted or unsubstituted C₂-C₆ alkynyl, substituted or unsubstituted C₃-C₈ cycloalkyl, substituted or unsubstituted five- to ten-membered aryl, or substituted or unsubstituted C₆-C₁₆ aralkyl —OR or —SR, where R denotes a substituted or unsubstituted C₁-C₆ alkyl, substituted or unsubstituted C₂-C₆ alkenyl, substituted or unsubstituted C₂-C₆ alkynyl, substituted or unsubstituted C₃-C₈ cycloalkyl, substituted or unsubstituted five- to ten-membered aryl, or substituted or unsubstituted C₆-C₁₆ aralkyl.

According to some embodiments, R⁶ is —H, -D, halogen, substituted or unsubstituted C₁-C₄ alkyl, substituted or unsubstituted C₂-C₄ alkenyl, substituted or unsubstituted C₂-C₄ alkynyl, substituted or unsubstituted C₃-C₆ cycloalkyl, substituted or unsubstituted five- or six-membered aryl, or substituted or unsubstituted C₆-C₁₀ aralkyl —OR or —SR, where R denotes a substituted or unsubstituted C₁-C₄ alkyl, substituted or unsubstituted C₂-C₄ alkenyl, substituted or unsubstituted C₂-C₄ alkynyl, substituted or unsubstituted C₃-C₆ cycloalkyl, substituted or unsubstituted five- or six-membered aryl, or substituted or unsubstituted C₆-C₁₀ aralkyl.

According to some embodiments, R^{6′} is —H, -D, halogen, substituted or unsubstituted C₁-C₆ alkyl, substituted or unsubstituted C₂-C₆ alkenyl, substituted or unsubstituted C₂-C₆ alkynyl, substituted or unsubstituted C₃-C₈ cycloalkyl, substituted or unsubstituted five- to ten-membered aryl, or substituted or unsubstituted C₆-C₁₆ aralkyl —OR or —SR, where R denotes a substituted or unsubstituted C₁-C₆ alkyl, substituted or unsubstituted C₂-C₆ alkenyl, substituted or unsubstituted C₂-C₆ alkynyl, substituted or unsubstituted C₃-C₈ cycloalkyl, substituted or unsubstituted five- to ten-membered aryl, or substituted or unsubstituted C₆-C₁₆ aralkyl.

According to some embodiments, R^{6′} is —H, -D, halogen, substituted or unsubstituted C₁-C₄ alkyl, substituted or unsubstituted C₂-C₄ alkenyl, substituted or unsubstituted C₂-C₄ alkynyl, substituted or unsubstituted C₃-C₆ cycloalkyl, substituted or unsubstituted five- or six-membered aryl, or substituted or unsubstituted C₆-C₁₀ aralkyl —OR or —SR, where R denotes a substituted or unsubstituted C₁-C₄ alkyl, substituted or unsubstituted C₂-C₄ alkenyl, substituted or unsubstituted C₂-C₄ alkynyl, substituted or unsubstituted C₃-C₆ cycloalkyl, substituted or unsubstituted five- or six-membered aryl, or substituted or unsubstituted C₆-C₁₀ aralkyl.

According to some embodiments, R⁶ is —H.

According to some embodiments, R^{6′} is —H.

According to some embodiments, R⁶ and R^{6′} are each —H.

According to some embodiments, R⁶ and R⁵ form together a substituted or unsubstituted C₃-C₆ cycloalkyl ring.

According to some embodiments, R⁶ and R^{6′} form together a substituted or unsubstituted C₃-C₆ cycloalkyl ring.

According to some embodiments, the compound is a compound of Formula II

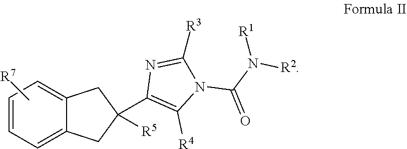

According to some embodiments, the compound is a compound of Formula III

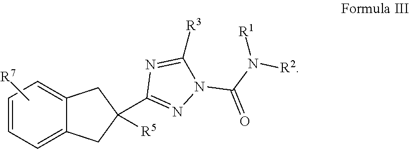

According to some embodiments, the compound is a compound of Formula IV

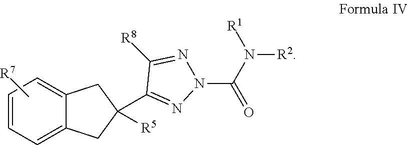

According to some embodiments, R³ is —H, -D, substituted or unsubstituted C₁-C₆ alkyl, substituted or unsubstituted C₂-C₆ alkenyl, substituted or unsubstituted C₂-C₆ alkynyl, substituted or unsubstituted C₃-C₈ cycloalkyl, substituted or unsubstituted five- to ten-membered aryl, or substituted or unsubstituted C₆-C₁₆ aralkyl, halogen, —NO₂, —CF₃, —OR or —SR, where R denotes a substituted or unsubstituted C₁-C₆ alkyl, substituted or unsubstituted C₂-C₆alkenyl, substituted or unsubstituted C₂-C₆ alkynyl, substituted or unsubstituted C₃-C₈ cycloalkyl, substituted or unsubstituted five- to ten-membered aryl, or substituted or unsubstituted C₆-C₁₆ aralkyl

According to some embodiments, R³ is —H, -D, substituted or unsubstituted C₁-C₄ alkyl, substituted or unsubstituted C₂-C₄ alkenyl, substituted or unsubstituted C₂-C₄ alkynyl, substituted or unsubstituted C₃-C₆ cycloalkyl, substituted or unsubstituted five- or six-membered aryl, or substituted or unsubstituted C₆-C₁₀ aralkyl, halogen, —NO₂, —CF₃, —OR or —SR, where R denotes a substituted or unsubstituted C₁-C₄ alkyl, substituted or unsubstituted C₂-C₄ alkenyl, substituted or unsubstituted C₂-C₄ alkynyl, substituted or unsubstituted C₃-C₆ cycloalkyl, substituted or unsubstituted five- or six-membered aryl, or substituted or unsubstituted C₆-C₁₀ aralkyl.

According to some embodiments, R³ is —H, -D, -Me, -Et, vinyl, ethynyl, —Pr, -iPr, halogen, —NO₂, or —CF₃.

According to some embodiments, R³ is —H.

According to some embodiments, R⁴ is —H, -D, substituted or unsubstituted C₁-C₆ alkyl, substituted or unsubstituted C₂-C₆ alkenyl, substituted or unsubstituted C₂-C₆ alkynyl, substituted or unsubstituted C₃-C₈ cycloalkyl, substituted or unsubstituted five- to ten-membered aryl, or substituted or unsubstituted C₆-C₁₆ aralkyl, halogen, —NO₂, —CF₃, —OR or —SR, where R denotes a substituted or unsubstituted C₁-C₆ alkyl, substituted or unsubstituted C₂-C₆alkenyl, substituted or unsubstituted C₂-C₆ alkynyl, substituted or unsubstituted C₃-C₈ cycloalkyl, substituted or unsubstituted five- to ten-membered aryl, or substituted or unsubstituted C₆-C₁₆ aralkyl

According to some embodiments, R⁴ is —H, -D, substituted or unsubstituted C₁-C₄ alkyl, substituted or unsubstituted C₂-C₄ alkenyl, substituted or unsubstituted C₂-C₄ alkynyl, substituted or unsubstituted C₃-C₆ cycloalkyl, substituted or unsubstituted five- or six-membered aryl, or substituted or unsubstituted C₆-C₁₀ aralkyl, halogen, —NO₂, —CF₃, —OR or —SR, where R denotes a substituted or unsubstituted C₁-C₄ alkyl, substituted or unsubstituted C₂-C₄ alkenyl, substituted or unsubstituted C₂-C₄ alkynyl, substituted or unsubstituted C₃-C₆ cycloalkyl, substituted or unsubstituted five- or six-membered aryl, or substituted or unsubstituted C₆-C₁₀ aralkyl.

According to some embodiments, R⁴ is —H, -D, -Me, -Et, vinyl, ethynyl, —Pr, -iPr, halogen, —NO₂, or —CF₃.

According to some embodiments, R⁴ is —H.

According to some embodiments, R⁵ is a substituted or unsubstituted C₁-C₆ alkyl, substituted or unsubstituted C₂-C₆ alkenyl, substituted or unsubstituted C₂-C₆ alkynyl, substituted or unsubstituted C₃-C₈ cycloalkyl, substituted or unsubstituted five- to ten-membered aryl, substituted or unsubstituted C₆-C₁₆ aralkyl, —OR or —SR, where R denotes a substituted or unsubstituted C₁-C₆ alkyl, substituted or unsubstituted C₂-C₆ alkenyl, substituted or unsubstituted C₂-C₆ alkynyl, substituted or unsubstituted C₃-C₈ cycloalkyl, substituted or unsubstituted five- to ten-membered aryl, or substituted or unsubstituted C₆-C₁₆ aralkyl.

According to some embodiments, R⁵ is a substituted or unsubstituted C₁-C₄ alkyl, substituted or unsubstituted C₂-C₄ alkenyl, substituted or unsubstituted C₂-C₄ alkynyl, substituted or unsubstituted C₃-C₆ cycloalkyl, substituted or unsubstituted five- or six-membered aryl, substituted or unsubstituted C₆-C₁₀ aralkyl, —OR or —SR, where R denotes a substituted or unsubstituted C₁-C₄ alkyl, substituted or unsubstituted C₂-C₄ alkenyl, substituted or unsubstituted C₂-C₄ alkynyl, substituted or unsubstituted C₃-C₆ cycloalkyl, substituted or unsubstituted five- or six-membered aryl, or substituted or unsubstituted C₆-C₁₀ aralkyl.

According to some embodiments, R⁵ is -Me, -Et, vinyl, ethynyl, —Pr, -iPr, -Bu, benzyl or phenethyl.

According to some embodiments, R⁵ is -Me.

According to some embodiments, R⁵ is -Et.

According to some embodiments, R⁷ represents one to four substituents on the aryl ring which are independently from each other selected from —H, -D, halogen, —NO₂, substituted or unsubstituted C₁-C₆ alkyl, substituted or unsubstituted C₂-C₆ alkenyl, substituted or unsubstituted C₂-C₆ alkynyl, substituted or unsubstituted C₃-C₆ cycloalkyl, substituted or unsubstituted five- to ten-membered aryl, or substituted or unsubstituted C₆-C₁₆ aralkyl, —OR and —SR groups, where R denotes a substituted or unsubstituted C₁-C₆ alkyl, substituted or unsubstituted C₂-C₆ alkenyl, substituted or unsubstituted C₂-C₆ alkynyl, substituted or unsubstituted C₃-C₆ cycloalkyl, substituted or unsubstituted five- to ten-membered aryl, or substituted or unsubstituted C₆-C₁6 aralkyl.

According to some embodiments, R⁷ represents one to four substituents on the aryl ring which are independently from each other selected from —H, -D, halogen, —NO₂, substituted or unsubstituted C₁-C₄ alkyl, substituted or unsubstituted C₂-C₄ alkenyl, substituted or unsubstituted C₂-C₄ alkynyl, substituted or unsubstituted C₃-C₆ cycloalkyl, substituted or unsubstituted five- or six-membered aryl, or substituted or unsubstituted C₆-C₁₀ aralkyl, —OR and —SR groups, where R denotes a substituted or unsubstituted C₁-C₄ alkyl, substituted or unsubstituted C₂-C₄ alkenyl, substituted or unsubstituted C₂-C₄ alkynyl, substituted or unsubstituted C₃-C₆ cycloalkyl, substituted or unsubstituted five- or six-membered aryl, or substituted or unsubstituted C₆-C₁₀ aralkyl.

According to some embodiments, R⁷ represents one to four substituents on the aryl ring which are independently from each other selected from —H, -Me, -Et, vinyl, ethynyl, —Pr, -iPr, -Bu, benzyl and phenethyl.

According to some embodiments, R⁷ is —H.

According to some embodiments, X is O and A is —CH₂—.

According to some embodiments, Y is C, Z is N, and W is C.

According to some embodiments, R³ is —H, R⁴ is —H, R⁵ is -Et, R⁶ is —H, R⁷ is —H, and R⁸ is —H.

According to some embodiments, the compound is selected from the group consisting of:
N-ethyl-4-(2-ethyl-2,3-dihydro-1H-inden-2-yl)-N-methyl-1H-imidazole-1-carboxamide;
4-(2-ethyl-2,3-dihydro-1H-inden-2-yl)-N,N-dimethyl-1H-imidazole-1-carboxamide;
N,N-diethyl-4-(2-ethyl-2,3-dihydro-1H-inden-2-yl)-1H-imidazole-1-carboxamide;
1-(dimethylcarbamoyl)-4-(2-ethylindan-2-yl)-3-methyl-1H-imidazol-3-ium trifluoromethanesulfonate;
4-(2-ethylindan-2-yl)-N,N-dimethyl-2H-1,2,3-triazole-2-carboxamide;
3-(2-ethylindan-2-yl)-N,N-dimethyl-1H-1,2,4-triazole-1-carboxamide;
N-benzyl-4-(2-ethylindan-2-yl)-N-methyl-1H-imidazole-1-carboxamide;
(4-(2-ethylindan-2-yl)-1H-imidazol-1-yl)(piperidin-1-yl)methanone;
4-(2-ethylindan-2-yl)-N,N-dimethyl-1H-imidazole-1-carbothioamide; and
N,N-diethyl-4-(2-(ethyl-d₅)indan-2-yl)-1H-imidazole-1-carboxamide.

According to some embodiments, the compound is selected from the group consisting of:
N-ethyl-4-(2-ethyl-2,3-dihydro-1H-inden-2-yl)-N-methyl-1H-imidazole-1-carboxamide;
4-(2-ethyl-2,3-dihydro-1H-inden-2-yl)-N,N-dimethyl-1H-imidazole-1-carboxamide;
N,N-diethyl-4-(2-ethyl-2,3-dihydro-1H-inden-2-yl)-1H-imidazole-1-carboxamide;
1-(dimethylcarbamoyl)-4-(2-ethylindan-2-yl)-3-methyl-1H-imidazol-3-ium
trifluoromethanesulfonate;
4-(2-ethylindan-2-yl)-N,N-dimethyl-2H-1,2,3-triazole-2-carboxamide; and
3-(2-ethylindan-2-yl)-N,N-dimethyl-1H-1,2,4-triazole-1-carboxamide.

According to some embodiments, the compound is selected from the group consisting of:
N-ethyl-4-(2-ethyl-2,3-dihydro-1H-inden-2-yl)-N-methyl-1H-imidazole-1-carboxamide;
4-(2-ethyl-2,3-dihydro-1H-inden-2-yl)-N,N-dimethyl-1H-imidazole-1-carboxamide; and
N,N-diethyl-4-(2-ethyl-2,3-dihydro-1H-inden-2-yl)-1H-imidazole-1-carboxamide.

According to some embodiments, the compound is a compound of Formula V

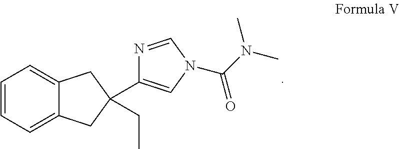 [SMILES: CCC1(c2cn(C(=O)N(C)C)cn2)Cc2ccccc2C1]

According to some embodiments, the compound is a compound of Formula VI

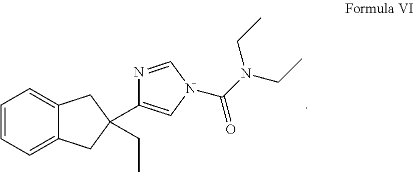 [SMILES: CCN(CC)C(=O)n1cnc(C2(CC)Cc3ccccc3C2)c1]

Generally, if any alkyl, alkenyl, alkynyl, cycloalkyl, aryl, aralkyl or heterocycle in any one of the R variables R¹, R², R³, R⁴, R⁵, R⁶, R^{6′}, R⁷ or R⁸ is substituted, it may be substituted with at least one substituent selected from deuterium, halogen, hydroxy, keto group, C₁-C₆ alkyloxy, including C₁-C₄ alkyloxy (such as methoxy or ethoxy), and C₁-C₆ alkylthio, including C₁-C₄ alkylthio.

A compound of formula I may form stable acid or basic salts, and in such cases administration of a compound as a salt may be appropriate, and pharmaceutically acceptable salts may be made by conventional methods such as those described below.

Examples of salts derived from pharmaceutically-acceptable inorganic bases include aluminum, ammonium, calcium, copper, ferric, ferrous, lithium, magnesium, manganic, manganous, potassium, sodium, zinc and the like. Salts derived from pharmaceutically-acceptable organic bases include salts of primary, secondary and tertiary amines, including substituted amines, cyclic amines, naturally-occurring amines and the like, such as arginine, betaine, caffeine, choline, N,N-dibenzylethylenediamine, diethylamine, 2-diethylaminoethanol, 2-dimethylaminoethanol, ethanolamine, ethylenediamine, dibenzylamine, morpholine, N-ethylmorpholine, N-methylpiperidine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine (i.e., 2-amino-2-hydroxymethylpropane-1,3-diol), tris-(2-hydroxyethyl)amine, and the like. When compounds of the present invention contain relatively basic functionalities, acid addition salts can be obtained by contacting the neutral form of such compounds with a sufficient amount of the desired acid, either neat or in a suitable inert solvent.

Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydroiodic, or phosphorous acids and the like, as well as the salts derived from relatively nontoxic organic acids like acetic, propionic, isobutyric, malonic, salicyclic, ascorbic, benzoic, succinic, suberic, fumaric, mandelic, phthalic, o-glycerophosphoric, benzenesulfonic, p-tolylsulfonic, citric, tartaric, methanesulfonic, and the like. Also included are salts of amino acids such as argininate and the like, and salts of organic acids like glucuronic or galactunoric acids and the like (see, for example, Berge, S. M., et al, “Pharmaceutical Salts”, Journal of Pharmaceutical Science, 1977, 66, 1-19). A preferred pharmaceutically-acceptable salt is the hydrochloride salt.

It will be appreciated by those skilled in the art that the compounds of the present invention can exists as stereoisomers (for example, optical (+ and −), geometrical (cis and trans) and conformational isomers (axial and equatorial). All such stereoisomers are included in the scope of the present invention.

It will be appreciated by those skilled in the art that the compounds of the invention can contain a chiral center. The compounds may thus exist in the form of two different optical isomers (i.e. (+) or (−) enantiomers). All such enantiomers and mixtures thereof including racemic mixtures are included within the scope of the invention. The single optical isomer or enantiomer can be obtained by method well known in the art, such as chiral HPLC, enzymatic resolution and chiral auxiliary.

In case of formula III and IV, only one regioisomer is shown. However, the invention also relates to other regioisomers, that is, if the azole ring is carbamoylated at another ring nitrogen than the one shown in the formulas.

It is also to be understood that compounds of the invention and salts thereof can exist in solvated as well as unsolvated forms such as, for example, hydrated forms.

### Therapeutic Uses

Another aspect of the invention refers to a compound of the invention for use in medicine.

Another aspect of the invention refers to a compound of the invention for use in the treatment of a neurodegenerative disease. Preferably, the neurodegenerative disease is Alzheimer's disease, Parkinson's disease, or amyotrophic lateral sclerosis.

It is noted that any of the compounds of the present invention can be used separately or in combination, particularly as adjuvant therapy administered simultaneously, alternatively or successively with respect to a first-line therapy suitable for the treatment of a neurodegenerative disease, such as Alzheimer's disease, Parkinson's disease and amyotrophic lateral sclerosis.

Another aspect of the invention provides a pharmaceutical composition which comprises a compound according to the invention as described above, or a pharmaceutically acceptable salt thereof, and a pharmaceutically acceptable carrier, adjuvant or vehicle.

The present invention thus provides pharmaceutical composition comprising a compound of this invention, or a pharmaceutically acceptable salt or stereoisomers thereof together with a pharmaceutically acceptable carrier, adjuvant, or vehicle, for administration to a patient, such as a human patient.

Examples of pharmaceutical compositions include any solid (tablets, pills, capsules, granules etc.) or liquid (solutions, suspensions or emulsions) composition. In a preferred embodiment, the pharmaceutical compositions are in oral form, either solid or liquid. Suitable dose forms for oral administration may be tablets, capsules, syrups or solutions and may contain conventional excipients known in the art such as binding agents, for example syrup, acacia, gelatin, sorbitol, tragacanth, or polyvinylpyrrolidone: fillers, for example lactose, sugar, maize starch, calcium phosphate, sorbitol or glycine; tabletting lubricants, for example magnesium stearate; disintegrants, for example starch, polyvinylpyrrolidone, sodium starch glycollate or microcrystalline cellulose; or pharmaceutically acceptable wetting agents such as sodium lauryl sulfate.

The solid oral compositions may be prepared by conventional methods of blending, filling, or tabletting. Repeated blending operations may be used to distribute the active agent throughout those compositions employing large quantities of fillers. Such operations are conventional in the art. The tablets may for example be prepared by wet or dry granulation and optionally coated according to methods well known in normal pharmaceutical practice, in particular with an enteric coating.

The pharmaceutical compositions may also be adapted for parenteral administration, such as sterile solutions, suspensions or lyophilized products in the appropriate unit dosage form. Adequate excipients can be used, such as bulking agents, buffering agents or surfactants.

The mentioned formulations will be prepared using standard methods such as those described or referred to in the European and US Pharmacopoeias and similar reference texts.

Administration of the compounds or compositions of the present invention may be by any suitable method, such as intravenous infusion, oral preparations, and intraperitoneal and intravenous administration Oral administration is preferred because of the convenience for the patient and the chronic character of the diseases to be treated.

Generally, an effective administered amount of a compound of the Invention will depend on the relative efficacy of the compound chosen, the severity of the disorder being treated and the weight of the sufferer. However, active compounds will typically be administered once or more times a day for example 1, 2, 3 or 4 times daily, with typical total daily doses in the range of from 0.1 to 1000 mg/kg/day.

The compounds and compositions of this invention may be used with other drugs to provide a combination therapy. The other drugs may form part of the same composition or be provided as a separate composition for administration at the same time or at different time.

Another aspect of the invention refers to a composition of the invention for use in medicine.

Another aspect of the invention refers to a composition of the invention for use in the treatment of a neurodegenerative disease. Preferably, the neurodegenerative disease is Alzheimer's disease, Parkinson's disease, or amyotrophic lateral sclerosis.

Another aspect of the invention refers to the use of a compound of the invention in the manufacture of a medicament, such as a medicament for the treatment of a neurodegenerative disease. Preferably, the neurodegenerative disease is Alzheimer's disease, Parkinson's disease, or amyotrophic lateral sclerosis.

Processes In a further aspect, the present invention provides a process of preparing a compound of formula I.

If not commercially available, the necessary starting materials for the procedures such as those described below may be made by procedures which are selected from standard organic chemistry techniques, techniques which are analogous to the synthesis of known structurally similar compounds, or techniques, which are analogous to the procedures described in the examples.

It will also be appreciated that in some of the reactions mentioned herein it may be necessary/desirable to protect any sensitive groups in compounds. The instances where protection is necessary or desirable are known to those skilled in the art, as are suitable methods for such protection.

Specifically, the present invention provides a process for the preparation of a compound of the invention, the process comprising reacting a compound of the formula VII

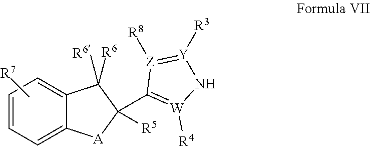

or a regioisomer thereof with a compound of formula VIII

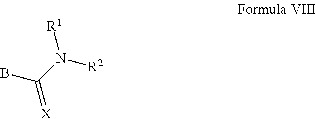

in the presence of a suitable base, such as an inorganic or organic base (e.g., triethylamine, DIPEA, pyridine, N-methylimidazole, LDA, potassium carbonate, sodium carbonate, sodium hydroxide, potassium hydroxide, potassium tert-butoxide, etc.);
wherein R¹, R², R³, R⁴, R⁵, R⁶, R^{6′}, R⁷, R⁸, X, Y, Z and W are as defined herein, and wherein B represents a suitable leaving group, such as halogen (F, Cl, Br) or N-alkylimidazolium.

In a further aspect the present invention provides a synthetic route for 4-(2-ethylindan-2-yl)-1H-imidazole (INN name: atipamezole) that was not previously described. The enolate of an alkyl ester of indane-2-carboxylic acid is C-alkylated with iodoethane or bromoethane at low temperatures to afford alkyl 2-ethylindane-2-carboxylate. The ester is then hydrolyzed to the 2-ethylindane-2-carboxylic acid. A Weinreb amide is prepared from 2-ethylindane-2-carboxylic acid chloride, reduced to the corresponding aldehyde, which is then converted to 4-(2-ethylindan-2-yl)-1H-imidazole in a two-step van Leusen reaction.

The present invention thus further provides a process for the preparation of 4-(2-ethylindan-2-yl)-1H-imidazole, which comprises reacting 2-ethylindane-2-carbaldehyde with arylsulfonylmethyl isocyanide (preferably toluenesulfonylmethyl isocyanide), followed by reaction with ammonia.

The present invention further provides a process for the preparation of 2-ethylindane-2-carbaldehyde, which comprises reacting 2-ethyl-N-methoxy-N-methylindan-2-carboxamide with lithium aluminium hydride or diisobutylaluminium hydride.

The present invention further provides a process for the preparation of 2-ethyl-N-methoxy-N-methylindan-2-carboxamide, which comprises reacting the acid chloride of 2-ethylindane-2-carboxylic acid with N,O-dimethylhydroxylamine hydrochloride in the presence of a base (e.g., triethylamine, DIPEA, pyridine, N-methylimidazole, potassium carbonate, sodium carbonate, sodium hydrogencarbonate, potassium tert-butoxide, etc.).

### EXAMPLES

### Chemistry—General Information

The reagents and solvents were used as received from commercial suppliers. Tetrahydrofuran (THF) was distilled from sodium/benzophenone and stored under Ar over 4 Å molecular sieves prior to use. After extraction, organic phases were dried over anhydrous sodium sulfate.

Reactions were monitored using analytical thin-layer chromatography (TLC) on silica gel 60 F₂₅₄ Al plates. Developed plates were inspected under UV light and, if necessary, visualized with ninhydrin, vanillin/sulfuric acid, Dragendorff's or potassium permanganate stains. Melting points were determined with Büchi 535 Melting Point Apparatus (uncorrected). Nuclear magnetic resonance spectra were recorded on a Bruker Avance III 400 MHz spectrometer at 400 MHz for ¹H, 100 MHz for ¹³C, and 376 MHz for ¹⁹F nucleus, respectively, using CDCl₃ with TMS as the internal standard as solvent. Chemical shifts are reported in parts per million (ppm), TMS peak was calibrated to 0 ppm or, alternatively, the central peak of the residual solvent resonance was used as the internal standard—i.e., 7.27 ppm for ¹H and 77.16 ppm for ¹³C, respectively. ¹⁹F spectra were not calibrated. The multiplicities are reported as follows: s (singlet), d (doublet), t (triplet), q (quartet), m (multiplet), dd (doublet of doublets), ddd (doublet doublet of doublets), td (triplet of doublets), qd (quartet of doublets), and br (broad), number of equivalent nuclei (by integration), coupling constants (J) quoted in Hertz (Hz). Mass spectra were recorded on Thermo Scientific Q Executive Plus LC-MS/MS spectrometer and IR spectra on Thermo Nicolet FT-IR spectrophotometer. Column chromatography was performed on silica gel (Silica gel 60, particle size: 0.035-0.070 mm, Merck). UPLC analyses were performed on Thermo Scientific Dionex UltiMate 3000 modular system (Thermo Fisher Scientific Inc.). The general method used a Waters Acquity UPLC® HSS C18 SB column (2.1×50 mm, 1.8 μm) thermostated at 40° C., with: injection volume, 5 μL; sample, 0.1-0.2 mg/mL in MeOH; flow rate, 0.4 mL/min; detector k, 220 and 254 nm; mobile phase A: 0.1% TFA (v/v) in water; mobile phase B: MeCN. Gradient: 0-5 min, 20-100% B; 5-6 min, 20% B. For aqueous stability study, injection volume 5 μL and detection at 220 nm were used. The purities of the tested compounds were established to be ≥95%, as determined by UPLC.

General procedure—RP-CC: Compounds were purified by reversed-phase column chromatography (RP-CC) (Isolera Biotage One Flash Chromatography system, Biotage® Sfar C18 Duo 100 Å 30 m column, 30 g) using a gradient of 0.1% TFA in deionized water and MeCN as eluent (gradient 0-100% MeCN in 6 column volumes (300 mL); 100% MeCN for 2 column volumes (100 mL)). After the RP-CC, fractions containing the product were combined and organic volatiles were evaporated in vacuo. The remaining aqueous solution was made alkaline (pH 8) with 1 M NaOH_{(aq)} and extracted with DCM (2×30 mL). The combined organic phase was dried over anhydrous sodium sulfate, filtered, and volatile components evaporated in vacuo to afford pure product.

### Example 1: 4-(2-ethylindan-2-yl)-1H-imidazole (6)

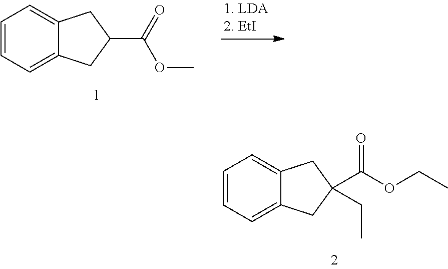 [SMILES: COC(=O)C1Cc2ccccc2C1 ; CCOC(=O)C1(CC)Cc2ccccc2C1]

Diisopropyl amine (1.25 eq., 4.6 mL) in anhydrous THF (20 mL) under Ar was cooled to −80° C., n-butyllithium (1.2 eq., 12.5 mL, 2.5 M in hexanes) was added dropwise, then the mixture was stirred at −10° C. for 10 min, cooled back to −80° C., and ethyl indane-2-carboxylate (4944 mg, 26.0 mmol) in THF (10 mL) was added dropwise. After stirring at −80° C. for 1 h, iodoethane (1.5 eq., 3.2 mL) was added, and the reaction mixture was left to warm up to room temperature during 4 h. 2 M hydrochloric acid (25 mL) was added, the solvent removed in vacuo, the residue extracted with diethyl ether (2×30 mL), organic phase separated, dried with sodium sulfate, and the solvent removed in vacuo to afford crude ethyl 2-ethylindane-2-carboxylate, which was sufficiently pure to be directly used in the next step. Yield: 4779 mg (21.9 mmol, 84%) of brownish oil. ESI-HRMS: m/z=219.1372 (MH⁺); C₁₄H₁₉O₂ requires: m/z=219.1380 (MH⁺). νₘₐₓ 2966 (m), 2935 (w), 1726 (vs), 1460 (m), 1219 (m), 1177 (m), 1110 (m), 1032 (m), 862 (w), 739 (s) cm⁻¹. ¹H NMR (400 MHz, CDCl₃) δ=0.89 (t, J=7.4, 3H), 1.26 (t, J=7.1, 3H), 1.78 (q, J=7.4, 2H), 2.90 (d, J=16.0, 2H), 3.46 (d, J=16.0, 2H), 4.16 (q, J=7.1, 2H), 7.11-7.19 (m, 4H). ¹³C NMR (101 MHz, CDCl₃) δ=9.92, 14.43, 31.67, 41.82, 54.79, 60.71, 124.57, 126.55, 141.63, 176.97.

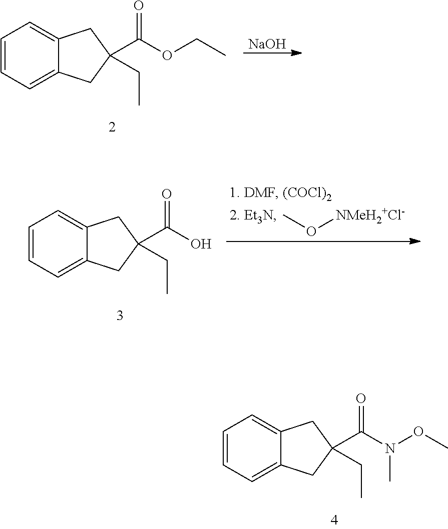

Ethyl 2-ethylindane-2-carboxylate 2 (3298 mg, 15.1 mmol) was dissolved in EtOH/H₂O (1:1, 50 mL+50 mL), sodium hydroxide (5.0 eq., 3021 mg) was added, and the mixture stirred at 80° C. for 48 h. The ethanol was removed in vacuo, the residue diluted with water (50 mL), extracted with diethyl ether (2×20 mL, discarded), acidified with concentrated hydrochloric acid to pH 1, and extracted with diethyl ether (2×30 mL). The organic phase was dried with sodium sulfate, filtered, and solvent removed in vacuo to afford crude 2-ethylindane-2-carboxylic acid 3 (Roeda, D.; Sipilä, H. t.; Bramoulld, Y.; Enas, J. D.; Vaufrey, F.; Dollé, F.; Crouzel, C. J. Label. Compd. Radiopharm. 2002, 45 (1), 37-47). Yield: 2635 mg (13.9 mmol, 92%) of brownish solid. νₘₐₓ 3022 (w), 2939 (w), 2852 (w), 2733 (w), 2643 (w), 1695 (vs), 1412 (m), 1338 (m), 1267 (s), 1183 (m), 942 (m), 733 (s) cm⁻¹. ¹H NMR (400 MHz, CDCl₃) δ=0.95 (t, J=7.4, 3H), 1.83 (q, J=7.4, 2H), 2.93 (d, J=16.1, 2H), 3.50 (d, J=16.1, 2H), 7.14-7.20 (m, 4H). ¹³C NMR (101 MHz, CDCl₃) δ=9.98, 31.53, 41.69, 54.81, 124.57, 126.68, 141.38, 183.74.

2-Ethylindane-2-carboxylic acid 3 (2635 mg, 13.9 mmol) was azeotropically dried with toluene (30 mL), dissolved in DCM (10 mL), DMF (100 L) was added, then oxalyl chloride (2.5 eq., 3 mL) was added dropwise under Ar, and the reaction mixture stirred at room temperature for 2 h. The volatiles were removed in vacuo, the residue dissolved in DCM (20 mL), cooled in an icebath, N,O-dimethylhydroxylamine hydrochloride (2.0 eq., 2702 mg) and triethylamine (3.5 eq., 6.8 mL) were added, and stirred at room temperature for 12 h. 2 M hydrochloric acid (40 mL) was added, extracted with DCM (30 mL), aqueous phase discarded, organic phase extracted with 1 M sodium hydroxide (20 mL, discarded), dried with sodium sulfate, filtered, and solvent removed in vacuo. 2-Ethyl-N-methoxy-N-methylindan-2-carboxamide 4 was isolated by column chromatography on silica (1. PE/EtOAc=5:1; 2. PE/EtOAc=2:1). Yield: 2205 mg (9.45 mmol, 68%) of beige oil. νₘₓ 2963 (m), 2938 (m), 2878 (w), 1647 (vs), 1459 (s), 1364 (s), 1297 (m), 1175 (m), 1009 (m), 746 (s) cm. ¹H NMR (400 MHz, CDCl₃) δ=0.86 (t, J=7.5, 3H), 1.76 (q, J=7.5, 2H), 2.99 (d, J=16.0, 2H), 3.24 (s, 3H), 3.47 (d, J=16.1, 2H), 3.73 (s, 3H), 7.12-7.21 (m, 4H).

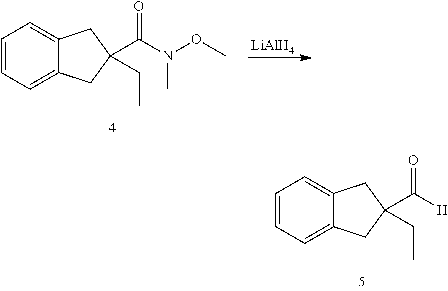

2-Ethyl-N-methoxy-N-methylindane-2-carboxamide 4 (2075 mg, 8.89 mmol) was azeotropically dried with toluene (30 mL), dissolved under Ar in anhydrous THF (10 mL), and cooled in an icebath. LiAlH₄ (0.5 eq., 1.9 mL, 2.4 M in THF) was added dropwise with stirring, and the stirring continued in an icebath for 1 h. 10% citric acid was added (30 mL), the solvent removed in vacuo, and the residue extracted with diethyl ether (2×20 mL). The organic phase was dried with sodium sulfate, filtered, and solvent removed in vacuo to afford crude 2-ethylindan-2-carbaldehyde 5 (John-Campbell, S. S.; Bull, J. A. Chem. Commun. 2019, 55 (62), 9172-9175). Yield: 1350 mg (7.75 mmol, 87%) of beige oil, which was immediately used further in the next step. ¹H NMR (400 MHz, CDCl₃) δ=0.90 (t, J=7.5, 3H), 1.82 (q, J=7.5, 2H), 2.85 (d, J=16.0, 2H), 3.33 (d, J=15.9, 2H), 7.13-7.20 (m, 4H), 9.60 (s, 1H). ¹³C NMR (101 MHz, CDCl₃) δ=9.67, 28.31, 38.53, 58.79, 124.72, 126.88, 141.12, 204.37.

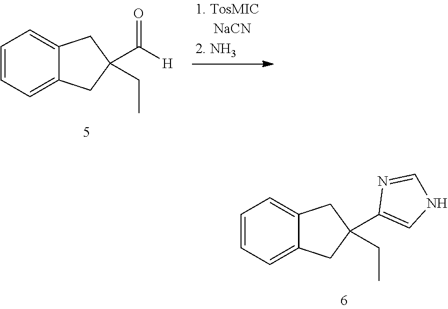 [SMILES: CCC1(C=O)Cc2ccccc2C1 ; CCC1(c2c[nH]cn2)Cc2ccccc2C1]

2-Ethylindane-2-carbaldehyde 5 (1430 mg, 8.21 mmol) was dissolved in absolute EtOH (8 mL), TosMIC (1.0 eq., 1602 mg) and NaCN (0.1 eq., 40 mg) were added, and the mixture stirred at room temperature for 12 h. The solvent was removed in vacuo, methanolic ammonia (25 mL, 4 M) was added, and stirred at 80° C. for 48 h in a pressure tube. The solvent was removed in vacuo, the residue dissolved in diethyl ether (30 mL), filtered, 5 mL of 2 M ethereal HCl were added, the suspension sonicated for 2 min, the supernatant discarded, and the precipitate washed with diethyl ether (15 mL). 2 M sodium hydroxide (10 mL) was added, extracted with DCM (2×30 mL), organic phase dried with sodium sulfate, the solvent removed in vacuo, and 4-(2-ethylindan-2-yl)-1H-imidazole 6 (INN name atipamezole (Parhi, S.; Karjalainen, A. US2008275252A1, Nov. 6, 2008; Karjalainen, A. J.; Virtanen, R. E.; Karjalainen, A. L.; Kurkela, K. O. A. U.S. Pat. No. 4,689,339A, Aug. 25, 1987; Lusis, V.; Muceniece, D.; Reine, I.; Zandersons, A. US2011028733A1, Feb. 3, 2011; CN111925330A, Nov. 13, 2020; CN112225700A, Jan. 15, 2021)) isolated by column chromatography on silica (1. DCM/MeOH=50:1; 2. DCM/MeOH=15:1). Yield: 818 mg (3.86 mmol, 47%) of beige solid. mp 123.2-124.4° C. ESI-HRMS: m/z=213.1378 (MH⁺); C₁₄H₁₇N₂ requires: m/z=213.1386 (MH⁺). νₘₐₓ 3069 (s), 2967 (s), 2914 (s), 2851 (vs), 2642 (br), 1668 (w), 1572 (w), 1473 (s), 1458 (vs), 1303 (s), 939 (m), 833 (s), 739 (s), 629 (s) cm-1. Purity: UPLC (254 nm): tᵣ=1.740 min, 99.0% total area. ¹H NMR (400 MHz, CDCl₃) δ=0.83 (t, J=7.4, 3H), 1.92 (q, J=7.3, 2H), 3.11 (d, J=15.6, 2H), 3.34 (d, J=15.6, 2H), 6.82 (d, J=0.9, 1H), 7.13-7.17 (m, 2H), 7.18-7.22 (m, 2H), 7.59 (d, J=0.9, 1H), 11.25 (s, 1H). ¹³C NMR (101 MHz, CDCl₃) δ=9.93, 33.19, 44.63, 47.69, 116.99, 124.57, 126.33, 134.91, 142.54, 143.37.

### Example 2: 4-(2-Ethylindan-2-yl)-N,N-dimethyl-1H-imidazole-1-carboxamide (7)

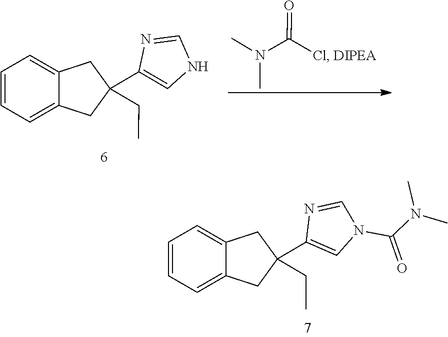

Atipamezole 6 (104 mg, 0.49 mmol), DIPEA (5.0 eq., 427 μL) and N,N-dimethylcarbamoyl chloride (5.0 eq., 226 μL) in MeCN (5 mL) were stirred under Ar at 60° C. for 6 h. The solvent was removed in vacuo and 4-(2-ethylindan-2-yl)-N,N-dimethyl-1H-imidazole-1-carboxamide 7 isolated by column chromatography on silica (1. PE/EtOAc=1:1; 2. EtOAc). Yield: 117 mg (0.41 mmol, 84%) of off-white solid. mp 121.1-122.2° C. ESI-HRMS: m/z=284.1746 (MH⁺); C₁₇H₂₂N₃O requires: m/z=284.1758 (MH⁺). νₘₐₓ 3111 (w), 2976 (w), 2959 (w), 2929 (w), 1672 (vs), 1478 (m), 1401 (s), 1257 (m), 1243 (m), 1188 (s), 1011 (s), 742 (s) cm⁻¹. Purity: UPLC (254 nm): tᵣ=2.073 min, 97.6%. ¹H NMR (400 MHz, CDCl₃) δ=0.74 (t, J=7.4, 3H), 1.89 (q, J=7.4, 2H), 3.00 (s, 6H), 3.03 (d, J=15.8, 2H), 3.24 (d, J=15.6, 2H), 6.89 (d, J=1.2, 1H), 7.05-7.09 (m, 2H), 7.12-7.15 (m, 2H), 7.80 (d, J=1.2, 1H). ¹³C NMR (101 MHz, CDCl₃) δ=9.84, 32.60, 38.31, 44.08, 48.37, 113.92, 124.41, 126.12, 136.47, 142.47, 148.77, 151.86.

### Example 3: N,N-diethyl-4-(2-ethylindan-2-yl)-1H-imidazole-1-carboxamide (8)

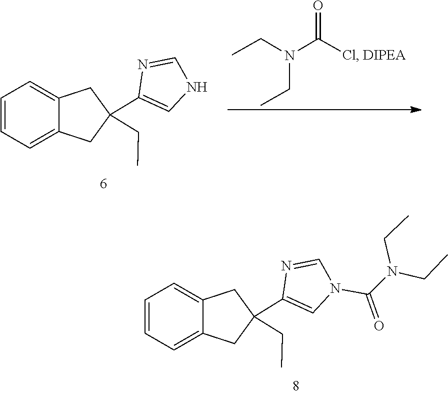

Atipamezole 6 (39 mg, 0.184 mmol), DIPEA (5.0 eq., 160 μL), and N,N-diethylcarbamoyl chloride (5.0 eq., 114 μL) in MeCN (3 mL) were stirred under Ar at 60° C. for 6 h. The solvent was removed in vacuo and N,N-diethyl-4-(2-ethylindan-2-yl)-1H-imidazole-1-carboxamide 8 isolated by column chromatography on silica (1. PE/EtOAc=2:1; 2. PE/EtOAc=1:1). Yield: 52 mg (0.167 mmol, 91%) of beige solid. mp 60.0-60.8° C. ESI-HRMS: m/z=312.2056 (MH⁺); C₁₉H₂₆N₃O requires: m/z=312.2070 (MH⁺). νₘₐₓ 3129 (w), 3107 (w), 2959 (m), 2929 (m), 1680 (vs), 1425 (s), 1360 (m), 1267 (s), 1218 (m), 1162 (m), 1027 (m), 952 (w), 855 (w), 752 (m), 744 (m), 674 (w), 629 (w) cm⁻¹. Purity: UPLC (220 nm): tᵣ=2.507 min, 99.7% total area. ¹H NMR (400 MHz, CDCl₃) δ=0.77 (t, J=7.4, 3H), 1.18 (t, J=7.1, 6H), 1.92 (q, J=7.4, 2H), 3.05 (d, J=15.6, 2H), 3.24 (d, J=15.6, 2H), 3.34 (q, J=7.1, 4H), 6.87 (d, J=1.3, 1H), 7.07-7.12 (m, 2H), 7.14-7.18 (m, 2H), 7.79 (d, J=1.3, 1H). ¹³C NMR (101 MHz, CDCl₃) δ=9.98, 13.21, 32.66, 42.63, 44.28, 48.58, 113.86, 124.53, 126.24, 136.15, 142.66, 148.92, 151.44.

### Example 4: 1-(Dimethylcarbamoyl)-4-(2-ethylindan-2-yl)-3-methyl-1H-imidazol-3-ium trifluoromethanesulfonate (9)

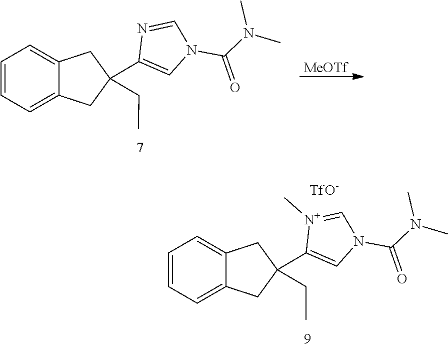 [SMILES: "MeOTf" ; CCC1(c2cn(C(=O)N(C)C)cn2)Cc2ccccc2C1 ; CCC1(c2cn(C(=O)N(C)C)c[n+]2C)Cc2ccccc2C1 ; "TfO-"]

4-(2-Ethylindan-2-yl)-N,N-dimethyl-1H-imidazole-1-carboxamide 7 (83 mg, 0.293 mmol) was azeotropically dried with toluene (10 mL), dissolved under Ar in anhydrous DCM (4 mL), and cooled in an icebath. Methyl triflate (1.5 eq., 50 L) was added and the mixture stirred at room temperature for 3 h. Diethyl ether (30 mL) was added, the mixture sonicated for 2 min, the supernatant decanted, the residue washed with diethyl ether (2×10 mL), and dried in vacuo. Yield: 116 mg (0.258 mmol, 89%) of beige semisolid. ESI-HRMS: m/z=298.1901 (MH⁺); C₁₈H₂₄N₃O requires: m/z=298.1914 (MH⁺). νₘₐₓ 3587 (br), 2963 (m), 2855 (w), 1735 (vs), 1486 (w), 1460 (w), 1390 (m), 1258 (vs), 1161 (s), 1031 (s), 638 (m) cm⁻¹. Purity: UPLC (220 nm): tᵣ=2.147 min, 95.1% total area. ¹H NMR (400 MHz, CDCl₃) δ=0.73 (t, J=7.3, 3H), 1.82 (q, J=7.2, 2H), 3.09 (s, 6H), 3.21 (d, J=15.6, 2H), 3.33 (d, J=15.6, 2H), 4.03 (s, 3H), 7.13-7.22 (m, 4H), 7.45 (d, J=1.3, 1H), 9.32 (s, 1H). ¹³C NMR (101 MHz, CDCl₃) δ=9.20, 30.47, 36.73, 38.73, 43.35, 46.52, 118.70, 119.07 (q, J=319.7, 320.7), 124.70, 127.17, 138.81, 139.95, 140.57, 147.41. ¹⁹F NMR (376 MHz, CDCl₃) δ=−78.49.

### Example 5: 5-(2-Ethylindan-2-yl)-1-methyl-1H-imidazole (10)

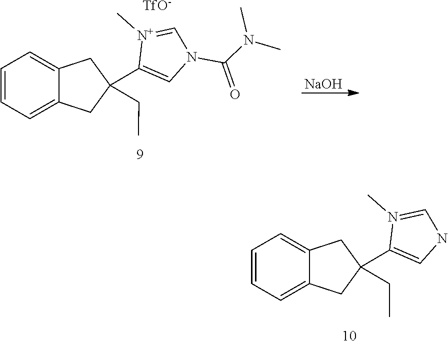

1-(Dimethylcarbamoyl)-4-(2-ethylindan-2-yl)-3-methyl-1H-imidazol-3-ium trifluoromethanesulfonate 9 (97 mg, 0.217 mmol) was dissolved in MeOH, 50% NaOH (100 μL) was added, and the mixture stirred for 4 h at room temperature. The solvent was removed in vacuo and 5-(2-ethylindan-2-yl)-1-methyl-1H-imidazole 10 isolated by column chromatography on silica (1. DCM/MeOH=20:1; 2. DCM/MeOH=5:1). Yield: 37 mg (0.164 mmol, 75%) of colourless oil. ESI-HRMS: m/z=227.1531 (MH⁺); C₁₅H₁₉N₂ requires: m/z=227.1543 (MH⁺). νₘₐₓ 3388 (br), 2962 (vs), 2932 (s), 2874 (m), 1637 (br), 1500 (vs), 1485 (s), 1458 (vs), 1378 (w), 1237 (s), 1120 (s), 912 (m), 816 (m), 743 (vs), 690 (m), 657 (s) cm⁻¹. Purity: UPLC (254 nm): tᵣ=1.943 min, 97.2% total area. ¹H NMR (400 MHz, CDCl₃) δ=0.68 (t, J=7.4, 3H), 1.73 (q, J=7.4, 2H), 3.15 (d, J=15.3, 2H), 3.31 (d, J=15.3, 2H), 3.69 (s, 3H), 6.81 (s, 1H), 7.12-7.17 (m, 2H), 7.18-7.23 (m, 2H), 7.35 (s, 1H). ¹³C NMR (101 MHz, CDCl₃) δ =9.40, 31.11, 33.44, 44.01, 46.38, 124.60, 126.56, 127.89, 136.90, 139.48, 141.62.

### Example 6: 4-(2-Ethylindan-2-yl)-2H-1,2,3-triazole (13)

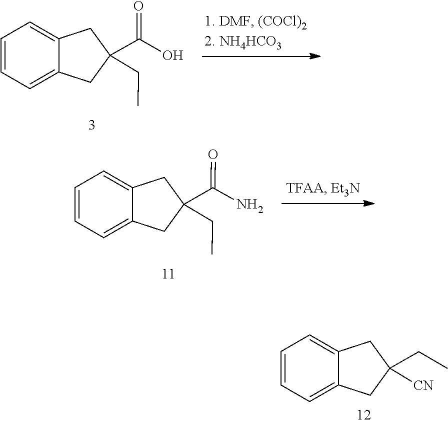

2-Ethylindane-2-carboxylic acid 3 (3100 mg, 16.3 mmol) was azeotropically dried with toluene (50 mL), dissolved in DCM (10 mL), DMF (100 μL) was added. Oxalyl chloride (3.0 eq., 4.2 mL) was added dropwise with stirring under Ar at room temperature. After 2 h, the volatiles were removed in vacuo, the residue redissolved in DCM (20 mL), ammonium bicarbonate (6.0 eq., 7.7 g) was added (gas effervescence!) and the mixture vigorously stirred at room temperature for 18 h. Water (50 mL) was then added, and 2-ethylindane-2-carboxamide 11 extracted into DCM (2×30 mL). The organic phase was dried with sodium sulfate, filtered, and the solvent removed in vacuo. The crude 2-ethylindane-2-carboxamide 11 (2015 mg, 10.65 mmol) was azeotropically dried with toluene (50 mL), dissolved in DCM (15 mL), triethylamine was added (4.0 eq., 6.7 mL), the mixture cooled in an icebath under Ar, and trifluoroacetic anhydride (2.0 eq., 2.96 mL) was added dropwise. After stirring for 2 h at room temperature, water (50 mL) and DCM (10 mL) were added, the organic phase successively extracted with 2 M hydrochloric acid (15 mL) and 1 M sodium hydroxide (20 mL), the solvent was removed in vacuo, and 2-ethylindane-2-carbonitrile 12 isolated by column chromatography on silica (1. PE; 2. PE/DCM=1:1). Yield: 1220 mg (7.13 mmol, 44% over three steps) of yellowish oil. νₘₐₓ 3269 (m), 2971 (m), 2845 (w), 2234 (w), 1722 (vs), 1639 (vs), 1496 (vs), 1465 (s), 1441 (s), 1237 (vs), 1155 (vs), 1024 (s), 762 (s) cm⁻¹. ¹H NMR (400 MHz, CDCl₃) δ=1.17 (t, J=7.4, 3H), 1.80 (q, J=7.4, 2H), 3.06 (d, J=15.7, 2H), 3.44 (d, J=15.7, 2H), 7.20-7.24 (m, 4H). ¹³C NMR (101 MHz, CDCl₃) δ=10.34, 31.23, 43.67, 43.78, 124.72, 124.82, 127.33, 139.56.

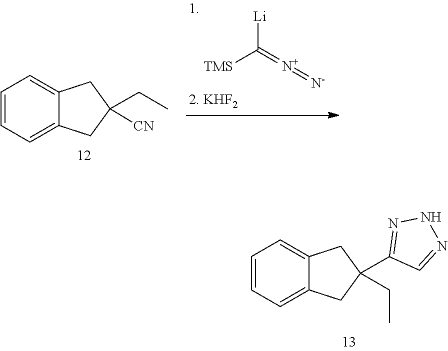

Trimethylsilyldiazomethane (1.6 eq., 5.43 mL, 2 M in hexanes) in anhydrous THF (5 mL) was cooled to −10° C. in a methanol-ice bath, n-butyllithium (1.6 eq., 4.3 mL, 2.5 M in hexanes) was added dropwise, and the mixture stirred for 30 min at this temperature. 2-Ethylindane-2-carbonitrile 12 (1162 mg, 6.79 mmol) in THF (3 mL) was then added and the reaction mixture left to warm up to room temperature within 4 h. Methanol (20 mL), potassium bifluoride (500 mg), and water (10 mL) were added, and the mixture stirred vigorously for 24 h. The solvent was removed in vacuo, the product extracted into diethyl ether (2×20 mL), and 4-(2-ethylindan-2-yl)-2H-1,2,3-triazole 13 isolated by column chromatography on silica (1. PE/EtOAc=5:1; 2. PE/EtOAc=2:1). Yield: 1191 mg (5.59 mmol, 82%) of yellowish semisolid. ESI-HRMS: m/z=214.1329 (MH⁺); C₁₃H₁₆N₃ requires: m/z=214.1339 (MH⁺). νₘₐₓ 3130 (s), 2962 (vs), 2935 (vs), 2844 (s), 1483 (m), 1458 (m), 744 (m) cm⁻¹. Purity: UPLC (220 nm): tᵣ=2.667 min, 98.0% total area. ¹H NMR (400 MHz, CDCl₃) δ=0.80 (t, J=7.4, 3H), 1.94 (q, J=7.4, 2H), 3.18 (d, J=15.6, 2H), 3.36 (d, J=15.6, 2H), 7.12-7.17 (m, 2H), 7.18-7.23 (m, 2H), 7.52 (s, 1H), 13.30 (s, 1H). ¹³C NMR (101 MHz, CDCl₃) δ=9.89, 33.52, 44.76, 47.12, 124.67, 126.63, 130.80, 142.07, 153.70.

### Example 7: 4-(2-Ethylindan-2-yl)-N,N-dimethyl-2H-1,2,3-triazole-2-carboxamide (14)

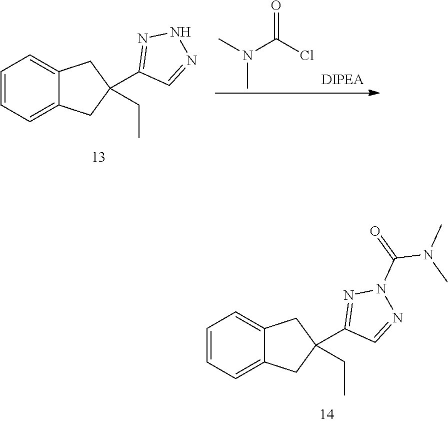

4-(2-Ethylindan-2-yl)-2H-1,2,3-triazole 13 (689 mg, 3.23 mmol), N,N-dimethylcarbamoyl chloride (5 eq., 1487 μL), and DIPEA (6 eq., 3.38 mL) in MeCN (10 mL) were stirred at 80° C. for 2 h. The solvent was removed in vacuo and 4-(2-ethylindan-2-yl)-N,N-dimethyl-2H-1,2,3-triazole-2-carboxamide 14 was isolated by RP-CC as the first of two barely resolved peaks.

Yield: 27 mg (0.0949 mmol, 2.9%) of beige oil. ESI-HRMS: m/z=285.1697 (MH⁺); C₁₆H₂₁N₄O requires: m/z=285.1710 (MH⁺). νₘₐₓ 3143 (w), 3022 (w), 2962 (m), 2934 (m), 2850 (w), 1704 (vs), 1482 (s), 1458 (s), 1405 (s), 1279 (m), 1254 (m), 1227 (m), 1155 (s), 1028 (s), 998 (s), 743 (s) cm⁻¹. Purity: UPLC (220 nm): tᵣ=3.207 min, 95.8% total area. ¹H NMR (400 MHz, CDCl₃) δ=0.79 (t, J=7.4, 3H), 2.00 (q, J=7.4, 2H), 3.12-3.19 (m, 5H), 3.31-3.39 (m, 5H), 7.11-7.15 (m, 2H), 7.16-7.20 (m, 2H), 7.85 (s, 1H). ¹³C NMR (101 MHz, CDCl₃) δ=9.94, 33.14, 38.56, 39.97, 44.67, 47.29, 122.01, 124.66, 126.57, 142.03, 149.62, 153.41.

### Example 8: 3-(2-Ethylindan-2-yl)-1H-1,2,4-triazole (15)

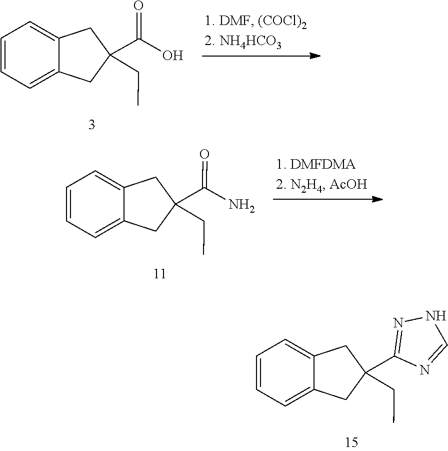 [SMILES: CCC1(C(=O)O)Cc2ccccc2C1 ; CCC1(C(N)=O)Cc2ccccc2C1 ; CCC1(c2nc[nH]n2)Cc2ccccc2C1]

2-Ethylindan-2-carboxylic acid 3 (648 mg, 3.4 mmol) was converted to carboxamide, as described under 13. The crude 2-ethylindane-2-carboxamide 11 and dimethylformamide dimethylacetal (DMFDMA, 10.0 eq., 4.53 mL) were stirred under Ar at 100° C. for 4 h. The volatiles were removed in vacuo, the residue was dissolved in glacial acetic acid (3 mL), hydrazine monohydrate (1.1 eq., 184 μL) was added, and the reaction mixture stirred at 60° C. for 12 h. 3-(2-ethylindan-2-yl)-1H-1,2,4-triazole 15 was isolated following RP-CC. Yield: 191 mg (0.896 mmol, 26% over four steps) of off-white solid. mp 127.5-128.7° C. ESI-HRMS: m/z=214.1330 (MH⁺); C₁₃H₁₆N₃ requires: m/z=214.1339 (MH⁺). νₘₐₓ 3114 (w), 3046 (w), 2956 (vs), 2844 (s), 2667 (br), 1500 (m), 1270 (s), 988 (s), 869 (s), 751 (vs) cm⁻¹. Purity: UPLC (254 nm): tᵣ=1.933 min, 95.3% total area. ¹H NMR (400 MHz, CDCl₃) δ=0.80 (t, J=7.4, 3H), 1.98 (q, J=7.3, 2H), 3.18 (d, J=15.8, 2H), 3.56 (d, J=15.8, 2H), 7.10-7.18 (m, 4H), 7.94 (s, 1H), 13.48 (br s, 1H). ¹³C NMR (101 MHz, CDCl₃) δ=9.86, 33.29, 43.74, 48.54, 124.61, 126.71, 141.48, 147.76, 165.34.

### Example 9: 3-(2-Ethylindan-2-yl)-N,N-dimethyl-1H-1,2,4-triazole-1-carboxamide (16)

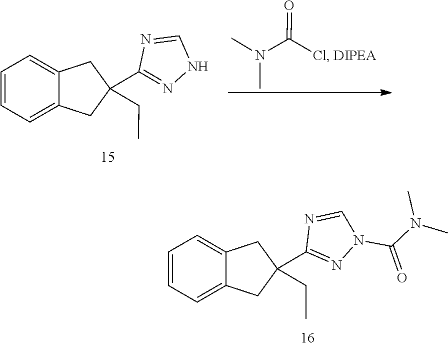

3-(2-Ethylindan-2-yl)-1H-1,2,4-triazole 15 (48 mg, 0.225 mmol), DIPEA (5.0 eq., 196 μL), and N,N-dimethylcarbamoyl chloride (5.0 eq., 104 μL) in MeCN (3 mL) were stirred under Ar for 6 h at 60° C. The solvent was removed in vacuo and 3-(2-ethylindan-2-yl)-N,N-dimethyl-1H-1,2,4-triazole-1-carboxamide 16 isolated by column chromatography on silica (1. PE/EtOAc=2:1; 2. EtOAc). Yield: 32 mg (0.113 mmol, 50%) of beige solid. mp 68.1-69.6° C. ESI-HRMS: m/z=285.1698 (MH⁺); C₁₆H₂₁N₄O requires: m/z=285.1710 (MH⁺). νₘₐₓ 3123 (w), 2959 (m), 2923 (m), 2850 (w), 1709 (vs), 1516 (s), 1481 (s), 1394 (s), 1270 (s), 1193 (s), 1002 (s), 743 (s), 694 (s) cm⁻¹. Purity: UPLC (220 nm): tᵣ=3.337 min, 96.7% total area. ¹H NMR (400 MHz, CDCl₃) δ=0.78 (t, J=7.4, 3H), 1.97 (q, J=7.4, 2H), 3.10 (d, J=15.7, 2H), 3.17 (s, 6H), 3.58 (d, J=15.7, 2H), 7.09-7.13 (m, 2H), 7.16-7.20 (m, 2H), 8.68 (s, 1H). ¹³C NMR (101 MHz, CDCl₃) δ=9.94, 32.87, 38.80 (br), 43.48, 49.43, 124.52, 126.39, 142.23, 146.74, 150.10, 170.09.

### Example 10: N-benzyl-4-(2-ethylindan-2-yl)-N-methyl-1H-imidazole-1-carboxamide (17)

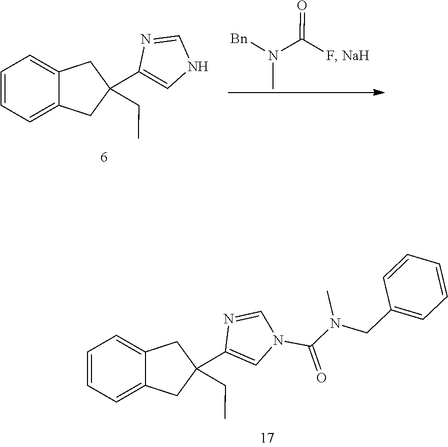

Atipamezole 6 (68 mg, 0.32 mmol), NaH (1.5 eq., 19 mg, 60 wt. % dispersion in mineral oil) and benzyl(methyl)carbamic fluoride (2.0 eq., 107 mg) in MeCN (3 mL) were stirred under Ar at 60° C. for 6 h. The solvent was removed in vacuo and N-benzyl-4-(2-ethylindan-2-yl)-N-methyl-1H-imidazole-1-carboxamide 17 isolated by column chromatography on silica (1. PE/EtOAc=3:1; 2. PE/EtOAc=1:1). Yield: 28 mg (0.078 mmol, 24%) of beige semisolid. ESI-HRMS: m/z=360.2063 (MH⁺); C₂₃H₂₆N₃O requires: m/z=360.2070 (MH⁺). ¹H NMR (400 MHz, CDCl₃) δ=0.76 (t, J=7.4, 3H), 1.91 (q, J=7.4, 2H), 2.98 (s, 3H), 3.03 (d, J=15.6, 2H), 3.22 (d, J=15.6, 2H), 4.57 (s, 2H), 6.93 (d, J=1.3, 1H), 7.07-7.16 (m, 4H), 7.22-7.26 (m, 2H), 7.31-7.41 (m, 3H), 7.85 (d, J=1.3, 1H). ¹³C NMR (101 MHz, CDCl₃) δ=9.99, 32.70, 36.47, 44.20, 48.58, 54.17, 114.11, 124.54, 126.25, 127.51, 128.15, 129.14, 135.55, 136.61, 142.59, 149.10, 152.29.

### Example 11: (4-(2-ethylindan-2-yl)-1H-imidazol-1-yl)(piperidin-1-yl)methanone (18)

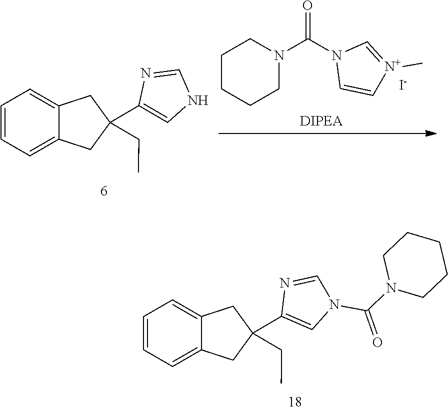 [SMILES: CCC1(c2c[nH]cn2)Cc2ccccc2C1 ; C[n+]1ccn(C(=O)N2CCCCC2)c1 ; [I-] ; CCC1(c2cn(C(=O)N3CCCCC3)cn2)Cc2ccccc2C1]

Atipamezole 6 (49 mg, 0.231 mmol), DIPEA (5.0 eq., 201 μL), and 3-methyl-1-(piperidine-1-carbonyl)-1H-imidazol-3-ium iodide (prepared in situ from (1H-imidazol-1-yl)(piperidin-1-yl)methanone and methyl iodide in acetonitrile; 1.04 mmol, 4.5 eq.) in MeCN (5 mL) were stirred under Ar at 60° C. for 4 h. The solvent was removed in vacuo and (4-(2-ethylindan-2-yl)-1H-imidazol-1-yl)(piperidin-1-yl)methanone 18 isolated by column chromatography on silica (1. PE/EtOAc=2:1; 2. PE/EtOAc=1:1). Yield: 29 mg (0.0897 mmol, 39%) of beige semisolid. ESI-HRMS: m/z=324.2065 (MH⁺); C₂₀H₂₆N₃O requires: m/z=324.2070 (MH⁺). ¹H NMR (400 MHz, CDCl₃) δ=0.77 (t, J=7.4, 3H), 1.57-1.64 (m, 4H), 1.64-1.71 (m, 2H), 1.91 (q, J=7.4, 2H), 3.05 (d, J=15.6, 2H), 3.25 (d, J=15.6, 2H), 3.43-3.48 (m, 4H), 6.84 (d, J=1.3, 1H), 7.08-7.12 (m, 2H), 7.14-7.19 (m, 2H), 7.77 (d, J=1.3, 1H). ¹³C NMR (101 MHz, CDCl₃) δ=10.00, 24.29, 25.85, 32.72, 44.27, 47.59, 48.56, 114.00, 124.56, 126.26, 136.49, 142.69, 149.04, 151.16.

### Example 12: 4-(2-ethylindan-2-yl)-N,N-dimethyl-1H-imidazole-1-carbothioamide (19)

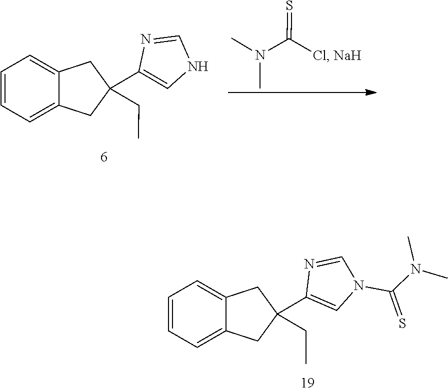

Atipamezole 6 (30 mg, 0.141 mmol), NaH (2.0 eq., 11 mg, 60 wt. % dispersion in mineral oil), and dimethylthiocarbamoyl chloride (2.0 eq., 35 mg) in MeCN (2 mL) were stirred under Ar at 60° C. for 4 h. The solvent was removed in vacuo and 4-(2-ethylindan-2-yl)-N,N-dimethyl-1H-imidazole-1-carbothioamide 19 isolated by column chromatography on silica (1. PE/EtOAc=3:1; 2. PE/EtOAc=1:1). Yield: 19 mg (0.0635 mmol, 45%) of yellowish semisolid. ¹H NMR (400 MHz, CDCl₃) δ=0.78 (t, J=7.4, 3H), 1.90 (q, J=7.4, 2H), 3.05 (d, J=15.6, 2H), 3.16-3.39 (m, 8H), 6.88 (d, J=1.3, 1H), 7.08-7.12 (m, 2H), 7.15-7.19 (m, 2H), 7.83 (d, J=1.3, 1H). ¹³C NMR (101 MHz, CDCl₃) δ=10.02, 32.65, 43.81, 44.22, 48.55, 115.39, 124.58, 126.29, 137.23, 142.62, 149.34, 179.76.

### Example 13: N,N-diethyl-4-(2-(ethyl-d₅)indan-2-yl)-1H-imidazole-1-carboxamide (21)

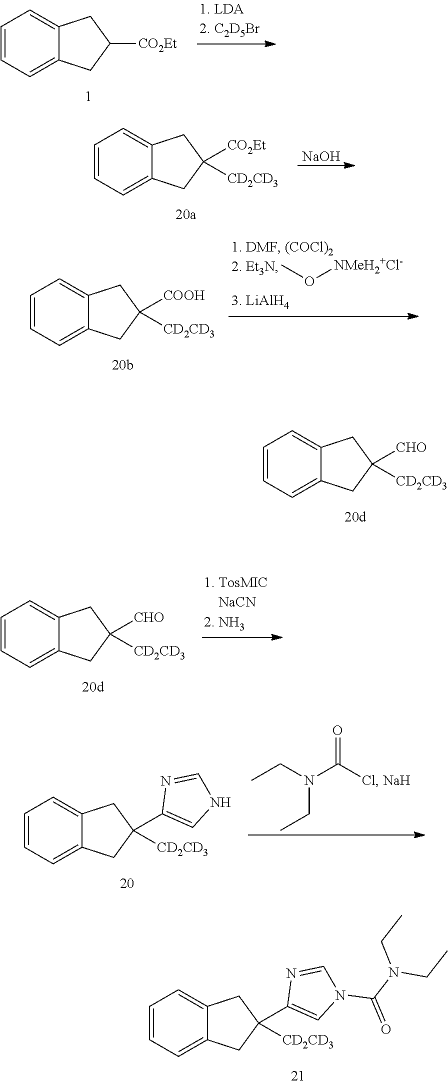

Diisopropyl amine (1.2 eq., 3.75 mL) in anhydrous THF (20 mL) under Ar was cooled to −80° C., n-butyllithium (1.15 eq., 10.3 mL, 2.5 M in hexanes) was added dropwise, then the mixture was stirred at 0° C. for 10 min, cooled back to −80° C., and ethyl indane-2-carboxylate (4244 mg, 22.3 mmol) in THF (10 mL) was added dropwise. After stirring at −80° C. for 1 h, bromoethane-d5 (1.2 eq., 2 mL, 99 atom % D) was added, and the reaction mixture was stirred at −80° C. for 2 h, and then left to warm up to room temperature during 4 h. 2 M Hydrochloric acid (25 mL) was added, the solvent removed in vacuo, the residue extracted with diethyl ether (2×30 mL), organic phase separated, dried with sodium sulfate, and the solvent removed in vacuo to afford crude ethyl 2-(ethyl-d₅)indane-2-carboxylate 20a (4660 mg) as yellowish oil, which was immediately used in the next steps as described under Example 1 to afford 2-(ethyl-d₅)indan-2-carboxylic acid 20b (3499 mg), 2-(ethyl-d₅)-N-methoxy-N-methylindan-2-carboxamide 20c (3035 mg), 2-(ethyl-d₅)indan-2-carbaldehyde 20d (2875 mg), and 4-(2-(ethyl-d₅)indan-2-yl)-1H-imidazole/atipamezole-d₅ 20 (346 mg). 20: ESI-HRMS: m/z=218.1700 (MH⁺); C₁₄H₁₂D₅N₂ requires: m/z=218.1700 (MH⁺). ¹H NMR (400 MHz, CDCl₃) δ=3.11 (d, J=15.6, 2H), 3.34 (d, J=15.6, 2H), 6.81 (d, J=1.0, 1H), 7.13-7.18 (m, 2H), 7.19-7.23 (m, 2H), 7.58 (d, J=1.0, 1H), 11.86 (br s, 1H). ¹³C NMR (101 MHz, CDCl₃) δ=8.88 (hept, J=19.1), 31.68-32.64 (m), 44.62, 47.52, 116.92 (br s), 124.57, 126.32, 134.94, 142.60, 143.54 (br s).

Atipamezole-d₅ 20 (106 mg, 0.488 mmol), NaH (1.5 eq., 29 mg, 60 wt. % dispersion in mineral oil), and N,N-diethylcarbamoyl chloride (1.5 eq., 93 μL) in MeCN (3 mL) were stirred under Ar at room temperature for 4 h. The solvent was removed in vacuo and N,N-diethyl-4-(2-(ethyl-d₅)indan-2-yl)-1H-imidazole-1-carboxamide 21 isolated by column chromatography on silica (1. PE/EtOAc=2:1; 2. PE/EtOAc=1:1). Yield: 40 mg (0.126 mmol, 26%) of white solid. ESI-HRMS: m/z=317.2379 (MH⁺); C₁₉H₂₁D₅N₃O requires: m/z=317.2384 (MH⁺). ¹H NMR (400 MHz, CDCl₃) δ=1.18 (t, J=7.1, 6H), 3.04 (d, J=15.6, 2H), 3.24 (d, J=15.6, 2H), 3.34 (q, J=7.1, 4H), 6.87 (d, J=1.2, 1H), 7.07-7.12 (m, 2H), 7.14-7.18 (m, 2H), 7.79 (d, J=1.3, 1H). ¹³C NMR (101 MHz, CDCl₃) δ=8.34-9.45 (m), 13.19, 31.62 (p, J=19.8), 42.60, 44.22, 44.26, 48.36, 48.56, 113.79, 113.83, 124.50, 126.21, 136.12, 142.64, 142.65, 148.90, 148.94, 151.42. ¹³C NMR (101 MHz, CDCl₃) δ=9.98, 13.21, 32.66, 42.63, 44.28, 48.58, 113.86, 124.53, 126.24, 136.15, 142.66, 148.92, 151.44.

### Pharmacological Evaluation

### Inhibition of Cholinesterases

The inhibitory potencies of the compounds against the ChEs were determined using the method of Ellman (Ellman, G. L.; Courtney, K. D.; Andres, V.; Featherstone, R. M. Biochem. Pharmacol. 1961, 7 (2), 88-95). The compound stock solutions in DMSO were incubated with Ellman's reagent and the ChEs (final concentrations: 370 μM Ellman's reagent, approximately 1 nM or 50 pM hBChE or hAChE, respectively) in 0.1 μM sodium phosphate pH 8.0 for 5 min at 20° C. For time-dependency measurements, the pre-incubation time was varied (1 min, 5 min, 15 min, 30 min or 60 min). The reactions were started by the addition of the substrate (final concentration, 500 μM butyrylthiocholine iodide or acetylthiocholine iodide for hBChE and hAChE, respectively). The final content of DMSO was always 1%. The increase in absorbance at 412 nm was monitored for 2 min using a 96-well microplate reader (Synergy HT, BioTek Instruments, VT, USA). The initial velocities in the presence (vi) and absence (vo) of the test compounds were calculated. The inhibitory potencies were expressed as the residual activities, according to RA=(vᵢ−b)/(vₒ−b), where b is the blank value using phosphate buffer without ChEs. For IC₅₀ determinations, at least seven different concentrations for each compound were used. The IC₅₀ values were obtained by plotting the residual ChE activities against the applied inhibitor concentrations, with the experimental data fitted to a four-parameter logistic function (GraphPad Prism 9.4). Tacrine and donepezil were used as positive controls.

### α_{2A} Binding Assay

The ligand binding assay on human α_{2A} adrenoreceptor from CHO cells using antagonist radioligand [³H]RX821002 was performed by Eurofins (assay number 13, study ID FR095-0034701). In each experiment the respective reference compound (yohimbine) was tested concurrently with the test compounds, and the data were compared with historical values determined at Eurofins. The experiment was accepted in accordance with Eurofins validation Standard Operating Procedure. Inhibition above 50% is considered to represent significant effects of the test compounds. The inhibition constants (Kᵢ) were calculated using the Cheng-Prusoff equation.

TABLE 1
The biological activities of the compounds at ChEs and α_{2A} adrenoreceptors.

| General structure: | | hBChE | hAChE | |
|---|---|---|---|---|
| 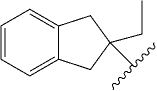 [SMILES: *C1(CC)Cc2ccccc2C1] | Com- pound | IC₅₀ ± SEM [nM]^{a} 5 min preincubation time-dependency observed: yes/no | | α_{2A}(h)^{b} inhibition at 1 μM [%] |
| 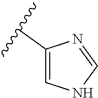 [SMILES: *c1c[nH]cn1] | 6 | Not active (78.7 ± 1.6%) | Not active (83.5 ± 2.4%) | 100.6 (Kᵢ = 0.77 nM) |
| 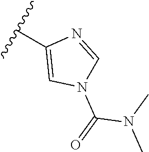 [SMILES: *c1cn(C(=O)N(C)C)cn1] | 7 | 12.6 ± 2.0 yes | 30507 ± 6304 yes | 6.0 |
| 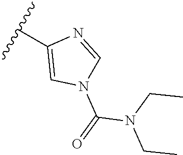 [SMILES: *c1cn(C(=O)N(CC)CC)cn1] | 8 | 1456 ± 218 yes | Not active (66.8 ± 4.7%) no | — |
| 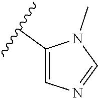 [SMILES: *c1cncn1C] | 10 | Not active (62.8 ± 2.6%) | Not active (75.3 ± 1.9%) | 14.8 |
| 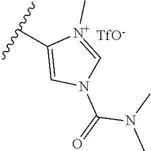 | 9 | 4.18 ± 0.77 yes | 430.6 ± 113.2 yes | — |
| 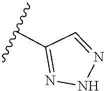 [SMILES: *c1cn[nH]n1] | 13 | Not active (60.3 ± 1.0%) | Not active (87.3 ± 7.5%) | 6.1 |
| 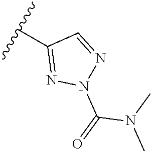 [SMILES: *c1cnn(C(=O)N(C)C)n1] | 14 | 2.39 ± 0.63 yes | 180.9 ± 35.5 yes | — |
| 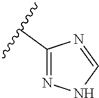 [SMILES: *c1nc[nH]n1] | 15 | Not active (95.9 ± 6.8%) | Not active (79.8 ± 7.0%) | 2.1 |
| 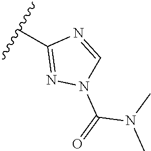 [SMILES: *c1ncn(C(=O)N(C)C)n1] | 16 | 4.12 ± 0.35 yes | 1981 ± 239 yes | — |
| 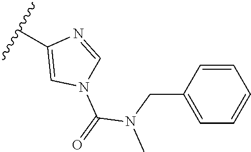 [SMILES: *c1cn(C(=O)N(C)Cc2ccccc2)cn1] | 17 | 95.72 ± 17.14 yes | 46.4 ± 3.3% yes | — |
| 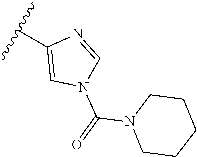 [SMILES: *c1cn(C(=O)N2CCCCC2)cn1] | 18 | 4.804 ± 0.944 yes | 5181 ± 1618 yes | — |
| 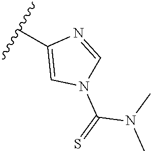 [SMILES: *c1cn(C(=S)N(C)C)cn1] | 19 | >100 000 yes | Not active (73.1 ± 4.5%) — | — |
| 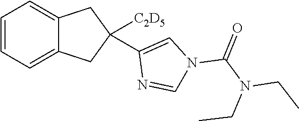 | 21 | 7829 ± 1319 yes | 58.9 ± 2.3% yes | — |

^{a}SEM-standard error of the mean, data are the average of two independent experiments, each performed in triplicate. For inactive compounds, RA-residual activity (mean ± standard deviation of one independent experiment performed in triplicate) is given in parentheses.
^{b}binding assay in human α_{2A} adrenoreceptor from CHO cells using antagonist radioligand [³H]RX821002. Inhibition higher than 50% is considered to represent significant effects of the test compounds.

Atipamezole's Kᵢ of 0.77 nM, obtained in CHO cells in competition with [³H]RX821002, agrees with the literature values: 1.2 nM for human α_{2A}-adrenoreceptors from CHO cells in competition with [³H]RS79948-197, and 1.6 nM for rat brain membranes in [³H]clonidine displacement assay (Vucicevic, J.; Srdic-Rajic, T.; Pieroni, M.; Laurila, J. M. M.; Perovic, V.; Tassini, S.; Azzali, E.; Costantino, G.; Glisic, S.; Agbaba, D.; Scheinin, M.; Nikolic, K.; Radi, M.; Veljkovic, N. Bioorg. Med. Chem. 2016, 24 (14), 3174-3183; Pertovaara, A.; Haapalinna, A.; Sirvia, J.; Virtanen, R. CNS Drug Rev. 2005, 11 (3), 273-288).

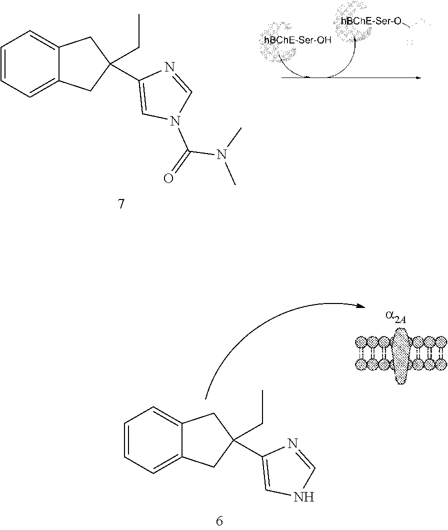 [SMILES: CCC1(c2cn(C(=O)N(C)C)cn2)Cc2ccccc2C1 ; CCC1(c2c[nH]cn2)Cc2ccccc2C1]

### Hydrolysis in Plasma

Samples were analysed by LC-MS system, which included Thermo Scientific UltiMate 3000 UHPLC liquid chromatograph and Thermo Scientific Exactive Plus Hybrid Quadrupole-Orbitrap mass spectrometer. The general method used a Waters Acquity UPLC® BEH C18 column (2.1×50 mm, 1.7 μm) and Waters Vanguard UPLC® BEH C18 precolumn (2.1×5 mm, 1.7 μm), thermostated at 40° C., with: injection volume, 1 μL; flow rate, 0.4 mL/min; autosampler temperature: 15° C., detector Full MS-SIM (runtime 1.5-3 min, positive mode, resolution: 70 000; AGC target: 3e⁶; maximum IT: 200 ms; scan range: 200-300 m/z); mobile phase A: 0.1% formic acid in MQ water; B: 0.1% formic acid in acetonitrile. Gradient: 0-3 min, 20-75% B; 3-3.1 min 75-95% B; 3.1-4 min 95% B; 4-4.1 min 95-20% B; 4.1-6 min 20% B. Processing: ICIS peak detection algorithm, analyte 6—expected retention time: 1.87 min, 30 s window, mass range 213.14; analyte 7—expected retention time: 2.40 min, 30 s window, mass range 284.18.

Compounds 6 and 7 were dissolved in MeOH to prepare 10 mM stock solutions that were further diluted with MeOH to 75 μM and 7.5 μM (working solutions). To construct the calibration curves, the highest concentration (800 nM) stock solution was prepared in plasma, and serially diluted with plasma to different compound concentrations (8, 20, 40, 60, 80, 200, 400, 600, 800 nM). 50 μL of the respective biological sample were diluted with 200 μL MeOH, vortexed, centrifuged at 4° C. and 5000 rpm for 10 min, the supernatant transferred to HPLC vials with inserts, and analysed. 5 replicates were injected to check the precision, and to ascertain the matrix effects in plasma, 0.5 μL of 75 μL 6 was diluted with 49.5 μL of either plasma or MQ water. Due to the mechanism of action (enzymatic hydrolysis), 7 itself is unstable in plasma—to construct the calibration curve, the BChE in plasma used was therefore first reversibly inhibited by a 5 min preincubation with 1 mM ethopropazine hydrochloride (from 50 mM stock in MeOH) and then used for dilutions. To monitor the progress of 7 hydrolysis in plasma, 1 vol. % of 7.5 μM 7 stock solution was mixed with plasma, 50 μL aliquots were drawn successively after 1, 5, and 60 min and analysed as described above. In a control experiment, the plasma used was pretreated for 5 min with 1 mM ethopropazine to inhibit BChE, then 7 was added, and the above procedure followed.

7 is readily hydrolysed by plasma BChE to 6—the fast reaction rate was evident by >15% product formed in the first minute (Table 2). Since the carbamoylated enzyme was most likely not regenerated during the monitoring time, the amount of liberated atipamezole was equimolar to the inactive, carbamoylated plasma BChE. As both 7 and BChE were being depleted, near the final timepoint the reaction rate slowed down considerably. The control sample with 1 mM ethopropazine (reversible selective BChE inhibitor) showed that still more than 10% of product was formed at the final timepoint. This can either indicate that BChE inhibition was still incomplete despite large excess of ethopropazine or that plasma enzymes other than BChE hydrolyse 7—the non-enzymatic hydrolysis was checked and found to be several orders of magnitude slower. 7 is readily hydrolysed in contact with plasma BChE—therefore, after an in vivo application (either parenteral or per os), adrenergic-active moiety 6 should be liberated in the bloodstream, permeate the blood-brain barrier, and reach the site of action in the central nervous system.

TABLE 2
Concentrations of reactant and product analytes
for the enzymatic hydrolysis of 7 in human plasma.

| | | | | nominal |
|---|---|---|---|---|
| | | concentration | | concentration: |
| | timepoint | [nM] | | 75 nM |
| sample | [min] | 6 | 7 | sum 6 + 7 |
| plasma | 1 | 15.911 | 66.508 | 82.419 |
| | 5 | 29.071 | 55.665 | 84.736 |
| | 60 | 65.375 | 22.431 | 87.806 |
| control | 1 | — | 74.477 | 74.477 |
| with 1 mM | 5 | — | 72.158 | 72.158 |
| ethopropazine | 60 | 9.816 | 71.131 | 80.947 |

1-44. (canceled)

45. A compound of Formula I: 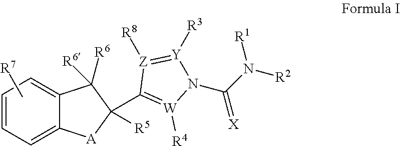
wherein:
R¹ and R², independently from each other, are a substituted or unsubstituted C₁-C₁₀ alkyl, substituted or unsubstituted C₂-C₁₀ alkenyl, substituted or unsubstituted C₂-C₁₀ alkynyl, substituted or unsubstituted C₃-C₁₀ cycloalkyl, substituted or unsubstituted five- to ten-membered aryl, or substituted or unsubstituted C₆-C₁₆ aralkyl; or R¹ and R², together with a nitrogen atom to which they are attached, form a substituted or unsubstituted three- to ten-membered heterocycle;
R³ and R⁴, independently from each other, are —H, -D, substituted or unsubstituted C₁-C₁₀ alkyl, substituted or unsubstituted C₂-C₁₀ alkenyl, substituted or unsubstituted C₂-C₁₀ alkynyl, substituted or unsubstituted C₃-C₁₀ cycloalkyl, substituted or unsubstituted five- to ten-membered aryl, or substituted or unsubstituted C₆-C₁₆ aralkyl, halogen, —NO₂, —CF₃, —OR, or —SR;
R⁵ is a substituted or unsubstituted C₁-C₁₀ alkyl, substituted or unsubstituted C₂-C₁₀ alkenyl, substituted or unsubstituted C₂-C₁₀ alkynyl, substituted or unsubstituted C₃-C₁₀ cycloalkyl, substituted or unsubstituted five- to ten-membered aryl, substituted or unsubstituted C₆-C₁₆ aralkyl, —OR, or —SR;
R⁶ and R^{6′}, independently from each other, are —H, -D, halogen, hydroxy, substituted or unsubstituted C₁-C₁₀ alkyl, substituted or unsubstituted C₂-C₁₀ alkenyl, substituted or unsubstituted C₂-C₁₀ alkynyl, substituted or unsubstituted C₃-C₁₀ cycloalkyl, substituted or unsubstituted five- to ten-membered aryl, substituted or unsubstituted C₆-C₁₆ aralkyl, —OR or —SR; or R⁶, together with R⁸ or R^{6′}, forms a substituted or unsubstituted C₃-C₆ cycloalkyl ring;
R⁷ is one to four substituents on the aryl ring which, independently from each other, are —H, -D, halogen, —NO₂, substituted or unsubstituted C₁-C₁₀ alkyl, substituted or unsubstituted C₂-C₁₀ alkenyl, substituted or unsubstituted C₂-C₁₀ alkynyl, substituted or unsubstituted C₃-C₁₀ cycloalkyl, substituted or unsubstituted five- to ten-membered aryl, substituted or unsubstituted C₆-C₁₆ aralkyl, —OR, or —SR;
X is O, S, or Se;
Y, Z, and W, independently from each other, are carbon, nitrogen, sulfur, or oxygen;
R⁸ is —H, -D, halogen, —NO₂, substituted or unsubstituted C₁-C₆ alkyl, substituted or unsubstituted C₂-C₆ alkenyl, substituted or unsubstituted C₂-C₆ alkynyl, substituted or unsubstituted C₃-C₅ cycloalkyl, substituted or unsubstituted five- to ten-membered aryl, substituted or unsubstituted C₆-C₁₆ aralkyl, —OR, or —SR;
A is —CH₂—, —CH₂CH₂—, —O—, —CH(—OH)—, —C(═O)—, —CHR—, —CR₂— —CH(—OR)—, —S—, —SO—, —SO₂—, or —N(R); and
R is a substituted or unsubstituted C₁-C₆ alkyl, substituted or unsubstituted C₂-C₆ alkenyl, substituted or unsubstituted C₂-C₆ alkynyl, substituted or unsubstituted C₃-C₆ cycloalkyl, substituted or unsubstituted five- to ten-membered aryl, or substituted or unsubstituted C₆-C₁₆ aralkyl;
or a pharmaceutically acceptable salt, racemate, diastereomer, enantiomer, regioisomer, hydrate, or solvate thereof.

46. The compound of claim 45, wherein R¹ is a substituted or unsubstituted C₁-C₆ alkyl, substituted or unsubstituted C₂-C₆ alkenyl, substituted or unsubstituted C₂-C₆ alkynyl, substituted or unsubstituted C₃-C₆ cycloalkyl, substituted or unsubstituted five- to ten-membered aryl, or substituted or unsubstituted C₆-C₁₆ aralkyl.

47. The compound of claim 45, wherein R¹ and R², independently from each other, are -Me, -Et, vinyl, ethynyl, —Pr, -iPr, -Bu, benzyl, or phenethyl.

48. The compound of claim 45, wherein Z is N and Y is C.

49. The compound of claim 45, wherein W is C or N.

50. The compound of claim 45, wherein W is C or N, Y is C, and Z is N.

51. The compound of claim 45, wherein A is —CH₂— and X is O.

52. The compound of claim 45, wherein the compound is of Formula II:

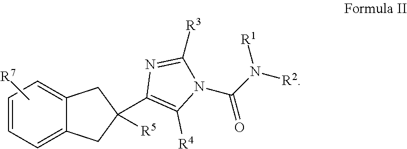

53. The compound of claim 45, wherein the compound is of Formula III:

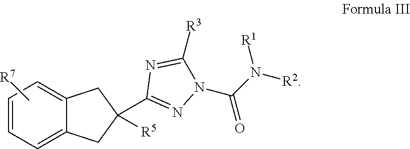

54. The compound of claim 45, wherein the compound is of Formula IV:

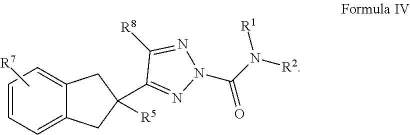

55. The compound of claim 45, wherein R³ is —H, -D, substituted or unsubstituted C₁-C₄ alkyl, substituted or unsubstituted C₂-C₄ alkenyl, substituted or unsubstituted C₂-C₄ alkynyl, substituted or unsubstituted C₃-C₆ cycloalkyl, substituted or unsubstituted five- or six-membered aryl, or substituted or unsubstituted C₆-C₁₀ aralkyl, halogen, —NO₂, or —CF₃, —OR, or —SR.

56. The compound of claim 45, wherein R⁴ is —H, -D, substituted or unsubstituted C₁-C₄ alkyl, substituted or unsubstituted C₂-C₄ alkenyl, substituted or unsubstituted C₂-C₄ alkynyl, substituted or unsubstituted C₃-C₆ cycloalkyl, substituted or unsubstituted five- or six-membered aryl, or substituted or unsubstituted C₆-C₁₀ aralkyl, halogen, —NO₂, —CF₃, —OR, or —SR.

57. The compound of claim 45, wherein R³ and R⁴ are each —H.

58. The compound of claim 45, wherein R⁵ is a substituted or unsubstituted C₁-C₆ alkyl, substituted or unsubstituted C₂-C₆ alkenyl, substituted or unsubstituted C₂-C₆ alkynyl, substituted or unsubstituted C₃-C₆ cycloalkyl, substituted or unsubstituted five- to ten-membered aryl, substituted or unsubstituted C₆-C₁₆ aralkyl, —OR, or —SR.

59. The compound of claim 45, wherein R⁵ is -Me or -Et.

60. The compound of claim 45, wherein the compound is selected from the group consisting of:
N-ethyl-4-(2-ethyl-2,3-dihydro-1H-inden-2-yl)-N-methyl-1H-imidazole-1-carboxamide;
4-(2-ethyl-2,3-dihydro-1H-inden-2-yl)-N,N-dimethyl-1H-imidazole-1-carboxamide;
N,N-diethyl-4-(2-ethyl-2,3-dihydro-1H-inden-2-yl)-1H-imidazole-1-carboxamide;
1-(dimethylcarbamoyl)-4-(2-ethylindan-2-yl)-3-methyl-1H-imidazol-3-ium trifluoromethanesulfonate;
4-(2-ethylindan-2-yl)-N,N-dimethyl-2H-1,2,3-triazole-2-carboxamide;
3-(2-ethylindan-2-yl)-N,N-dimethyl-1H-1,2,4-triazole-1-carboxamide;
N-benzyl-4-(2-ethylindan-2-yl)-N-methyl-1H-imidazole-1-carboxamide;
(4-(2-ethylindan-2-yl)-1H-imidazol-1-yl)(piperidin-1-yl)methanone;
4-(2-ethylindan-2-yl)-N,N-dimethyl-1H-imidazole-1-carbothioamide; and
N,N-diethyl-4-(2-(ethyl-d₅)indan-2-yl)-1H-imidazole-1-carboxamide.

61. The compound of claim 45, wherein the compound is selected from the group consisting of:
N-ethyl-4-(2-ethyl-2,3-dihydro-1H-inden-2-yl)-N-methyl-1H-imidazole-1-carboxamide;
4-(2-ethyl-2,3-dihydro-1H-inden-2-yl)-N,N-dimethyl-1H-imidazole-1-carboxamide; and
N,N-diethyl-4-(2-ethyl-2,3-dihydro-1H-inden-2-yl)-1H-imidazole-1-carboxamide.

62. A pharmaceutical composition comprising the compound of claim 45 and a pharmaceutically acceptable carrier, adjuvant, and/or vehicle.

63. The compound of claim 45, wherein the compound is operable for use in treatment of a neurodegenerative disease, the neurogenerative disease selected from the group consisting of: Alzheimer's disease, Parkinson's disease, and amyotrophic lateral sclerosis.

64. A process for preparation of the compound of claim 45, the process comprising reacting a compound of Formula VII: 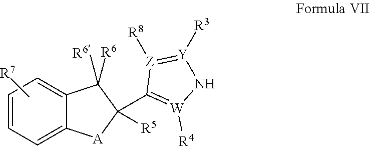
or a regioisomer thereof with a compound of Formula VIII: 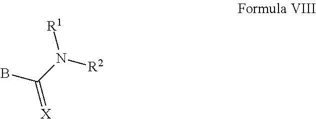
in a base, the base comprising at least one of: triethylamine, DIPEA, pyridine, N-methylimidazole, LDA, potassium carbonate, sodium carbonate, sodium hydroxide, potassium hydroxide, and potassium tert-butoxide; and
wherein B is a leaving group, the leaving group selected from a halogen or N-alkylimidazolium.
